SEQUENCE LISTING (22 sequences)

SEQ ID NO: 1 - protein, 630 aa, Synechocystis PCC6803
MLKLSWKSLLVSPAVIGAALVAGAASAAPDNVTNAQVLDQLDQYTAEGQSSAIDQVTSVSELRDVQPTAWAYEALKSLVERYGCIVGYPDRTFRGDRALSRWEFAAGLNACMNVMERLIQENVAVLREDIDKLKRLMQEFEAELAALGARVDNLEARTSFLEDHQFSTTTKLTGEVIFAPTAIFGTKKTLNNQTDNNQAVFQNRVRLQFNTSFSGEDLLVTRLAAGNGNRFKSFYPTQNVIDPETGNPIGIVGDYYESPTFTQVHQLSPGDNNNVAVDWLAYYVPLDLGENFRLNNYIAAWGGIWDDFVPTLNPYFDDYTGGKGSLSQFTAQNPIYSIGGGTGIGTSLELGFLSNLLGPTSLSLGYLASTGNNPSSGGSVTNPATGNNYDFSSGGNGLFNGGYSALAQITTNIFDRVSLGFTYVNAYTTPDAAIFGKGGTQGIVGTTAANLNRSELNDDFVNGLGVGAPPVDAATYNNNVSGGSTSGNVINPYDFGGKQTNSYGVQMAWNIADWLSFSAYGSYTNVTLIGKDNGDIWTYGGGFAFPDLGKEGNVLGIFAGVQPYVSGFTNNTLGTTFVTTANPLQVELFYKYQLTDNLSITPGVIWISKPEQTTNATDAFIGTVRGTFTF

SEQ ID NO: 2 - protein, 532 aa, Synechococcus sp. NIES-970
MSKAMRNLLSGATVALGATVAFSGAAIAEENQAVLDQISQYSTDNSVAQVNSVFQLSDVSPSDWAFDALRNLVENYNCIVGYPDGTFRGTRPLSRYEFAAGLNACLQQIERMLEGGGEVTQEDLAALRRLLNEFEAELATLGARVDDLDGRVEFLEDNQFSTTTKLNGEVIFNLGGAFGDSRADGPGPIDDEFTFSNRVRLNLDTSFTGRDRLRTRLEAGNITRLDRATGVDAARLGFDTNNGNDVEIGKLFYRFPVGDNITAYVGAVGLGLDDIFDVTNPYFESSGNGALSRFGRFNPIYRGPDGAGAGFNFDLGERVTFSAAYLADDGGASSPVSKNGLFNGNYTAGAQLDFAPSDNIMLSAVYTRRYQQGGDVNLSGSTGSDFAADPFGFAASSNNFGLQGSWLIGEKFNLGGWVGYTNADLKDPVVTGNADVWNWAVNAALLDVGGEGNTLGFIFGQPPKLTEADIDFGGGPITFDGSDSTYLAELQYRYKVNNNITITPGAYVLWNPNQNDANDTIVVGTVRTTFSF

SEQ ID NO: 3 - protein, 567 aa, Anabaena cylindrica PCC 7122
MSNLLWKSLVVSPAVLGAALLVSGTAIASPAAGNSASNAATEVSILETQAATEVVQQPVVLAQVKQDTQVLDQVKRYSNEGQNLQSQVTSVSQFSDVQPTDWAFQALQSLVERYGCIAGYPNGTYRGNRALTRYEFAAGLNACLDRVNELIATATADMVTKQDLATLQRLQEEFSAELATLRGRVDALEARTAELEANQFSTTTKLQGEAIFALTDAFNGDIAGKTVFQDRVRLNLQSSFTGRDMLNTRLAAGNAQNFGNTGESRQTFDVGSTGTNNVVLDKLTYEAPVGPAQVYIAATGGKHSHYAAVNNPYFFDKTDGGNGALSTFSSENPIYRIGGGSGIALNVPFGQGGGILKPSSLTLGYLASEANSPAAGEGLFDGNYAALGQLNFNVGNRIALAATYVHGYHGASSRLFDSGEGDDIVGTSLANNLSLGSASSSNSYGVSAAFRPSDKLSVSGFVSYHDITGFGAGDDYEAWSYGVGVALPDFGKKGNVLGIFGGAQPYSFNNNGGANSGDIPYQVEGFYKYRVSDNISVTPGVIWLPSIGQSSSSEDTFIGTLRTTFTF

SEQ ID NO: 4 - protein, 300 aa, Synechocystis PCC6803
MRVILCGYYGQDNAGDEALLVCLLQMLPATVEPVVLSANPQVTTARYGVKAHYNRDWGKIWQLLGQCDGFIWGGGSLMQDVTSVVSPLYYGGLMAIAQMRRLKTIAWAQGIGPLRRPPLRWFTQQVLRGCSGISVRDQASLQLVKQWGLKAFLAADPVWSLAAENASSSPSPLPMVAVNLRAHPLLTLERLAVITEALRDFQHQTQTHVRLIPLQKSQDLAIAEAIAVELPGSHEILYRPDPRECKGLFRGAEFTIGMRLHSLIMAAAEGSACFALSYDPKVSRLIAEVGLPGRELADLP

SEQ ID NO: 5 - protein, 339 aa, Synechocystis sp. PCC7942
MRALLCGYYGEGNGGDEALLAALLQLLPPTVEPLVLSGNPAATRTCYGVEACDRRSGAAVWQALRRSDLFIFGGGSLIQDVTSWTSPLYYCGLMGLAQQLGLKTIAWAQGIGPLQRKRTRWLARRTFQHCDRITVRDRGSAALLKEWGISVAIAPDPVWALEPLPLDQPLSNQEAIAVCLRPHPELTPERSDRLKAALTTLQSQTQAPILLIPFHHQQDRPLAAQWASEIPQAEVLDWQHPRQLLSYFQSVRLTIAMRFHGLVMAAAAGNRCFGLSYDPKVRRFLDALEQPGWDLPEIPDSAEAIAAAWLESWQAEPIHAAAIADLRQQVDVHRQALVF

SEQ ID NO: 6 - protein, 350 aa, Anabaena cylindrica PCC 7122
MVKIRALLSGYYGKGNGGDEALLATLLQMLPPDVTPVVLSGNPEETKKRYGVESYNRMAFLQIIKALRSCDAFIWGGGSLMQDATSSISPFYYGGLMALAQAMGLKTVAWGQGIGPLLRSQTRWLAKRNFALCTKISVRDRSSAALLSNWGIPHILAPDPVWALESKPVTELADLPKPRIAVILRNHPQLTETRLANLIHALVDLQQATQAFILLLPFQKSEDLDIAEKIQLQLKDVSKIICAEDPQLLKGIFRGVEMAIGMRLHSLIMAASEGCRCFALSYDPKVNHLMEDLEIPGWDLKDLPTDVDLMSKTWIQYYHNSEALSSEKIQSLVNGAFLHRDLLRASLSNK

SEQ ID NO: 7 - DNA, 1352 bp, Synechocystis PCC6803
atgcttaaactatcttggaagtccctgttagtgagccctgctgtgatcggtgccgcccttgtggctggtgctgctagtgctgctcccgacaacgttacgaatgctcaagttttagaccaattagaccaatacactgccgaaggtcagtcttctgccattgatcaagtaaccagcgttagcgaactccgcgatgtgcaacccactgcctgggcctatgaagcgctcaagagcttggtggaacgctacggttgtatcgttggttaccctgaccgtaccttccgtggtgatcgagccctcagccgttgggaatttgccgctggtcttaacgcctgtatgaacgttatggaacgtttgattcaggaaaacgtggctgttctccgggaggacatcgacaaactgaaacgcctcatgcaggaattcgaggcagaattggctgctttgggtgctcgggttgataacctcgaagcccgcacctctttcctcgaagaccatcagttttctaccaccaccaaattgaccggggaagtaatatttgctcccaccgccatttttggtaccaaaaagactctcaacaaccaaaccgataacaaccaagcggtattccagaaccgggttcgtttacagttcaacaccagcttctccggtgaagacctgttggtcactcgtctcgccgctggtaacggtaatcgcttcaagagcttctatcctactcagaatgttattgatcccgagaccggtaaccccatcggtattgtaggtgactactatgaaagccccacctttacccaggtgcaccagctttcccccggcgacaacaacaacgtagccgttgactggttagcttactatgttcccttggatctgggcgaaaacttccgtctcaacaactacattgctgcttggggcggtatctgggatgacttcgttcctaccctgaacccctatttcgacgactacactggcggcaaaggttccttgagtcagttcaccgcccagaaccccatctactccattggtggcggtactggtataggtacaagcctagagttgggcttcctcagcaatctgttgggccccacttccttgagcttgggttacttggctagcacgggcaacaaccccagcagtggtggttctgttactaaccctgccactggcaacaattatgacttcagcagcggtggtaacggtttgtttaacggtggctatagcgccttggctcaaattaccaccaacattttcgaccgggttagcctaggcttcacctacgttaatgcctacaccacccctgatgccgctatcttcggtaaaggtggcactcaaggtatcgtcggtaccactgcggctaacct

SEQ ID NO: 8 - DNA, 1353 bp, Synechococcus sp. NIES-970
atgtctaaagcaatgagaaatcttctcagtggtgcaaccgttgctctcggtgcaactgttgctttttctggcgcagcgatcgccgaagaaaaccaagccgtcctcgaccagatcagtcaatacagcactgataactccgttgctcaggtcaacagcgttttccagctcagcgacgtttccccttccgattgggcttttgacgcgctccgcaacttggttgaaaactacaactgtatcgttggttatcctgatggtactttccgtggcactcgccccctcagccgttatgagttcgccgcaggtctgaatgcttgtttgcaacagatcgaacggatgctcgaaggtggcggtgaagtaacccaagaagatctcgcagccctccgtcgtctgctcaacgaatttgaagcagaactagctaccctcggcgctcgtgttgatgaccttgatggccgtgttgagttccttgaagacaatcagttctccaccaccaccaaactgaatggggaagttatcttcaaccttggtggcgcttttggcgatagcagagctgatggtcctggcccaatcgatgatgagttcacattcagcaatcgtgttcgtctgaacctagacactagcttcactggtcgcgatcgcctccgcactcgtcttgaagctggcaatatcactcgcttagaccgtgcaactggtgtagatgcagcccgtctcggctttgacactaacaacggtaatgatgttgaaattgggaaattgttctaccgcttccctgtaggtgacaacattactgcctatgtcggtgccgttggcctaggtcttgatgacatatttgatgtaaccaacccatattttgagagcagcggtaatggtgccctctctcgtttcggtcgttttaaccccatctaccgtggccccgatggtgctggtgctggttttaacttcgatcttggtgaaagagttaccttctccgcagcttaccttgcggatgatggtggtgctagcagccctgtctcgaagaatggcctgttcaatggcaactacactgcgggtgctcagctcgattttgctccctctgacaacatcatgctgtctgccgtttatacccgtcgctaccaacagggtggcgatgtcaacctgagcggtagcactggtagtgattttgctgcagatccttttggttttgctgcttcttccaacaacttcggcttacaaggtagctggttgatcggtgaaaaattcaaccttggtggctgggtcggctacaccaatgctgatctcaaagatcctgttgtcaccggtaacgctgatgtttggaactgggctgttaacgccgctctacttgatgtcggcggcgaa

SEQ ID NO: 9 - DNA, 1360 bp, Anabaena cylindrica PCC 7122
atgtctaatctattgtggaaatccttggtggttagcccagccgtgttgggagccgccttattagtttcaggaacagcgatcgcatcgcccgccgcaggcaactcagcctcaaatgctgctactgaagtatcaatactagaaacacaagcggcaacagaggttgttcaacaacctgtagtattggctcaagtcaagcaagacactcaggttttagatcaagttaaacgctacagtaacgaaggtcaaaatctacaatctcaagtaacatcagtctctcagttttccgatgtccaaccaactgactgggcattccaagctttgcagtccttggtggaacgttatggttgtatcgcaggttatcccaatggtacatatcgcggaaaccgtgctttaacccgttatgaatttgccgctggtttgaatgcctgtttagaccgggtgaatgaattgattgccacagcaacagctgacatggtgacaaaacaggatttagccaccttacagcgcttgcaagaagaattttctgctgaattggcaactttacgcggtcgtgtcgatgctttggaagcacgcactgctgagttagaagctaatcaattctctactaccaccaaattgcaaggtgaagctatttttgctctgaccgatgcttttaatggtgacattgccggtaagaccgtttttcaagatagagtgcgtttgaacctgcaaagcagcttcacaggtcgagatatgttgaatacccgtttggctgcgggtaatgcacaaaactttggaaacactggggaaagtagacaaacctttgacgttggtagcactgggactaacaatgttgtgttagacaaactgacctatgaagctcctgttggtccagcacaggtctacatagcagctacaggtggtaaacacagccattatgctgctgttaacaacccttacttctttgataaaactgatggtggtaacggtgctttaagtacctttagttctgaaaaccccatctatcgaattggtggtggttctggtattgcccttaatgtaccctttggtcaaggtggcggtattctcaaaccaagttcattgacattgggttatttggcttctgaggctaatagtcctgctgctggtgaaggtttgttcgacggtaactatgctgctttaggtcagttaaactttaacgttggtaatcgcattgccttagctgctacttatgtacatggttatcatggtgctagtagccgtttgtttgactcaggagagggtgatgatatcgtaggtacatcactagctaataatctcagtttaggtagcgcctcgtccagcaactcctacggtgtttctgctgccttcagacctagcg

SEQ ID NO: 10 - DNA, 1032 bp, Synechocystis PCC6803
atgcgtgtcattctgtgtggttactacggccaagacaatgctggggatgaagcccttttagtctgtttgctgcaaatgcttcctgcgacggtggagccagtggtgctctccgccaatccccaggtcaccacagcgagatacggtgttaaagcccactacaaccgagactggggcaaaatttggcagcttttgggtcaatgtgacggttttatctgggggggaggcagtttgatgcaggatgtgaccagcgtggttagtcccctatactacggtggtttgatggcgatcgcccagatgcggcgtttaaaaaccattgcctgggcccagggcattggcccccttagaaggccgccgttgcgctggtttacccaacaggtgctccggggttgtagcggcatcagcgtacgggatcaagcttctttgcaattagttaaacaatggggcttaaaggcttttttagcggcggatccggtgtggtctttagccgctgaaaatgcgtcttcttcccccagccctttgcccatggtggcggtgaatttgcgggcccatcctttgttaaccttggaacgcttggcggtcattaccgaagccctaagggattttcaacaccagacccaaacccatgtgcggctcattcccctacaaaaatcccaggatttggccattgccgaagcgatcgccgtggaattgccggggagtcatgaaattctttaccgccccgaccccagggaatgtaaaggtttgttccggggggcggaattcaccattggcatgcggctccacagtttaatcatggccgcggcggagggcagtgcctgctttgcccttagttatgaccctaaagttagtcgtttaatcgcagaggtgggactaccgggccgggaattggcggatttacccagggatagcaacgaactcagccaggtttggcagggacatttccggcaaagacaaccctctaccggggtagagttcttgcaaaaatctgcccttcagcatcaaactttgttgcagaaaatttttgttgactag

SEQ ID NO: 11 - DNA, 1020 bp, Synechocystis sp. PCC7942
atgcgggcgttgctctgtggctattacggagagggcaatgggggagatgaggcgctgttggcagcgctgttgcaactcttgccgccgacagtggaaccgctggtgctgtcggggaatcctgcggcaactcgcacttgctacggcgtagaagcttgcgatcgccgatcgggggctgcggtttggcaggcactgcgacgcagtgatctattcatctttggcggcggcagcctgattcaagatgtcacaagctggacgagccctctctactactgcggtctgatggggctggcacaacagttaggtctcaaaacgatcgcctgggcccagggcattgggccactgcagcgcaaacgaacccgctggttggcacgccgaacgtttcagcactgcgatcgcattacagtgcgcgatcgcggttctgctgccttgctaaaagagtgggggatttctgtggcgattgccccggatcccgtttgggctttagagccgctgcctttagatcagccgctgtcgaaccaagaggcgatcgcggtttgtctgcggccccaccctgagttgacgcctgagcgcagcgatcgcttgaaagctgccctgacaactctgcaatcgcaaacccaagcacccatcctgctgattcccttccaccaccagcaagatcgaccactggccgcgcaatgggcaagcgaaattccccaagcagaagttttggactggcagcatccgcgccagctcctcagctactttcagtcggtgcgattaacgatcgccatgcgcttccatggattagtgatggcagcagcggctggaaatcgttgctttggcctcagctatgaccccaaagtgcgccgtttcctggatgcccttgagcaacccggctgggatttgccggagattccagactctgctgaggcgatcgcggccgcttggttagagtcttggcaagcagaaccgattcatgccgctgcgatcgcggacctccgtcagcaagtggatgtccatcgccaggctttagttttttga

SEQ ID NO: 12 - DNA, 1053 bp, Anabaena cylindrica PCC 7122
ctacttattactcaatgatgctctcaataaatcccgatgtaaaaaagctccatttactaaagattgtattttctcagatgataatgcttcgctattgtgataatattgaatccaggttttactcattaaatctacatcagttggtaaatcctttaaatcccatccaggtatttctaaatcttccatcaaatggttaaccttggggtcataactcaaagcaaaacaacgacaaccttcactagctgccataattaaactgtgtagacgcatccctattgccatttctacaccacgaaatataccttttaaaagttgtggatcttctgcacagataattttactaacatcttttaattgcagttgaattttttctgcaatatctaaatcttcacttttttgaaaaggcaataataaaataaatgcttgggtagcttgttgtaaatcaactaaggcatggattaaatttgctaaccgagtttctgtaagttggggatgatttcttaaaataacggcaattctaggttttggtaaatctgctaattctgttactggtttagattctaaagcccaaactggatcaggtgcaagaatatgaggtatgccccaatttgataataaagccgcactagagcgatcgcgtacactgattttagtacaaagagcaaaatttcgctttgctaaccaacgagtttgcgatcgcaataaaggccctattccctgtccccaagctacagttttcaaacccatcgcttgagctaaagccattaatcccccataataaaaagggctaatgctgctagtcgcatcctgcattaaacttccaccaccccagatgaaagcatcacaggaacgcaaagctttgataatctgcaaaaacgccatacggttgtaactttccacaccataacgctttttagtttcctcaggattcccagaaagcaccacaggcgttacatcaggtggtagcatttgcagaagtgtcgccaacaaagcttcatcaccaccattacctttaccgtaatacccagacaataacgcccgtatcttcaccat

SEQ ID NO: 13 - DNA, 40 bp, Artificial Sequence
  Synthesized Primer
cagtgaattcgagctcggtatatagcgttgcagtccctgg

SEQ ID NO: 14 - DNA, 40 bp, Artificial Sequence
  Synthesized Primer
gattacgccaagcttgcatgaccgcggtcacttcataacc

SEQ ID NO: 15 - DNA, 47 bp, Artificial Sequence
  Synthesized Primer
cagtgaattcgagctcggtaataaccgttgtcccttttgtttcatcg

SEQ ID NO: 16 - DNA, 45 bp, Artificial Sequence
  Synthesized Primer
tgttagtgagccctgctgttagctcccagtatctctatcactgat

SEQ ID NO: 17 - DNA, 51 bp, Artificial Sequence
  Synthesized Primer
acagcagggctcactaacagttttagagctagaaatagcaagttaaaataa

SEQ ID NO: 18 - DNA, 44 bp, Artificial Sequence
  Synthesized Primer
gattacgccaagcttgcatggggaacaagctgaatctgggcatc

SEQ ID NO: 19 - DNA, 45 bp, Artificial Sequence
  Synthesized Primer
ttttagtctgtttgctgcatagctcccagtatctctatcactgat

SEQ ID NO: 20 - DNA, 51 bp, Artificial Sequence
  Synthesized Primer
tgcagcaaacagactaaaagttttagagctagaaatagcaagttaaaataa

SEQ ID NO: 21 - DNA, 19 bp, Synechocystis PCC6803
acagcagggctcactaaca

SEQ ID NO: 22 - DNA, 19 bp, Synechocystis PCC6803
tgcagcaaacagactaaaa

(19) United States
(12) Patent Application Publication
(10) Pub. No.: US 2022/0325312 A1
(43) Pub. Date: Oct. 13, 2022

(54) MODIFIED CYANOBACTERIUM, MODIFIED CYANOBACTERIUM PRODUCTION METHOD, AND PROTEIN PRODUCTION METHOD

(71) Applicant: Panasonic Intellectual Property Management Co., Ltd., Osaka (JP)

(72) Inventor: Seiji KOJIMA, Nara (JP)

(73) Assignee: Panasonic Intellectual Property Management Co., Ltd., Osaka (JP)

(21) Appl. No.: 17/748,617

(22) Filed: May 19, 2022

Related U.S. Application Data
(63) Continuation of application No. PCT/JP2020/042549, filed on Nov. 16, 2020.

(30) Foreign Application Priority Data
Nov. 21, 2019 (JP) 2019-210114

(51) Int. Cl. C12P 21/02; C12N 1/20; C12N 15/74; C12N 15/11; C12N 9/22

(52) U.S. Cl. CPC C12P 21/02; C12N 1/20; C12N 15/74; C12N 15/11; C12N 9/22; C12N 2800/101; C12N 2310/20; C12N 2800/80

(57) ABSTRACT

A modified bacterium in which a function of a protein involved in binding between an outer membrane and a cell wall of cyanobacterium is suppressed or lost.

### CROSS REFERENCE TO RELATED APPLICATIONS

This is a continuation application of PCT International Application No. PCT/JP2020/042549 filed on Nov. 16, 2020, designating the United States of America, which is based on and claims priority of Japanese Patent Application No. 2019-210114 filed on Nov. 21, 2019. The entire disclosures of the above-identified applications, including the specifications, drawings and claims are incorporated herein by reference in their entirety.

### FIELD

The present disclosure relates to a modified cyanobacterium having improved secretory productivity of protein that is produced within the bacterial cell and secreted to the outside of the bacterial cell, a modified cyanobacterium production method, and a protein production method.

### BACKGROUND

Bio-manufacturing schemes that do not depend on fossil fuel and have low environmental load have been desired for a wide range of industrial fields from chemical industry to agricultural, fishery, and livestock industry. Bio-manufacturing using microbes has received attention as production systems that satisfy the requirements described above, because it can be performed in an environment of ambient pressure and temperature and has become able to produce a wide range of compounds by recent development of genetic manipulation technology. Among others, photosynthetic microbes such as cyanobacterium and algae can utilize carbon dioxide (CO₂) in air as a carbon source with light as an energy source and are therefore particularly expected as carbon-neutral next-generation bio-manufacturing systems.

For example, ethanol (Non Patent Literature (NPL) 1), isobutanol (NPL 2), alkanes (Patent Literature (PTL) 2), fatty acid (PTL 1), and protein (NPL 3) have been reported as materials that are produced using cyanobacterium.

For example, NPL 4 discloses an approach for allowing cyanobacterium to express protein.

### CITATION LIST

### Patent Literature

PTL 1: Japanese Patent No. 6341676
PTL 2: Japanese Unexamined Patent Application Publication No. 2015-109828

### Non Patent Literature

NPL 1: Jason Dexter and Pengcheng Fu, “Metabolic engineering of cyanobacteria for ethanol production”, Energy & Environmental Science, Royal Society of Chemistry, 2009, Vol. 2, pp. 857-864

NPL 2: Shota Atsumi et al., “Direct photosynthetic recycling of carbon dioxide to isobutyraldehyde”, Nature Biotechnology, Nature Publishing Group, 2009, Vol. 27, No. 12, pp. 1177-1180

NPL 3: Jie Zhou et al., “Discovery of a super-strong promoter enable efficient production of heterologous proteins in cyanobacteria”, Scientific Reports, Nature Research, 2014, Vol. 4, Article No. 4500

NPL 4: Andrew H. Ng et al., “Fine-Tuning of Photoautotrophic Protein Production by Combining Promoters and Neutral Sites in the Cyanobacterium Synechocystis sp. Strain PCC 6803”, Applied and Environmental Microbiology, American Society for Microbiology, 2015, Vol. 81, pp. 6857-6863

### SUMMARY

### Technical Problem

However, in the aforementioned conventional techniques, it is difficult to produce proteins efficiently using cyanobacteria.

In view of this, the present disclosure provides a modified cyanobacterium having improved protein secretory productivity, a modified cyanobacterium production method, and a protein production method using a modified cyanobacterium.

### Solution to Problem

A modified cyanobacterium according to an aspect of the present disclosure is a modified cyanobacterium in which a function of a protein involved in binding between an outer membrane and a cell wall of cyanobacterium is suppressed or lost.

### Advantageous Effects

The modified cyanobacterium and the modified cyanobacterium production method are capable of providing a modified cyanobacterium having improved protein secretory productivity. Furthermore, the protein production method according to the present disclosure is capable of efficiently producing protein.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram schematically illustrating a cell surface of a cyanobacterium.

FIG. 2 is a transmission electron microscope observation image of an ultrathin section of a modified cyanobacterium of Example 1.

FIG. 3 is an enlarged image of broken line region A of FIG. 2.

FIG. 4 is a transmission electron microscope image of an ultrathin section of a modified cyanobacterium of Example 2.

FIG. 5 is an enlarged image of broken line region B of FIG. 4.

FIG. 6 is a transmission electron microscope image of an ultrathin section of a modified cyanobacterium of Comparative Example 1.

FIG. 7 is an enlarged view of broken line region C of FIG. 6.

FIG. 8 is a graph illustrating the amount of protein (n=3, error bar=SD) in the culture supernatant of a modified cyanobacterium in Example 1, Example 2, and Comparative Example 1.

### DESCRIPTION OF EMBODIMENTS

### (Underlying Knowledge Forming Basis of Present Disclosure)

Cyanobacterium (also called blue-green bacterium or blue-green alga), a group of eubacterium, produces oxygen by splitting water through photosynthesis, and fixes CO₂ in air. Cyanobacterium can also fix nitrogen (N₂) in air, depending on its species. Rapid growth and high light use efficiency are known as characteristics of cyanobacterium. In addition, its genetic manipulation is easier than that of other eukaryotic alga species. Therefore, the utilization of cyanobacterium among the photosynthetic microbes is under active research and development. As mentioned above, the production of fuel such as ethanol (NPL 1), isobutanol (NPL 2), alkanes (PTL 2), and fatty acid (PTL 1) has been reported as examples of bio-manufacturing using cyanobacterium.

The production of materials serving as nutrient sources for organisms using cyanobacterium is also under research and development. For example, since protein can be synthesized only by living organisms, photosynthetic microbes which utilize light energy and CO₂ in the atmosphere are considered useful as an organism species for use in convenient and efficient protein production.

For example, NPL 3 describes a protein production method using a cyanobacterium Synechocystis sp. PCC6803 strain. The literature discloses the nucleotide sequence of a promoter for activating the gene transcription of an ethylene-forming enzyme with high efficiency, and a ribosomal binding sequence for enhancing translation.

NPL 4 discloses, in the case of expressing a recombinant protein with a Synechocystis sp. PCC6803 strain as a host, an approach for inserting a gene encoding the protein to a NS-pCC2 region on a plasmid retained by this strain. Use of the approach has been reported to improve the expression efficiency of the recombinant protein by approximately 14 times as compared with the case of inserting the gene onto chromosomal DNA (deoxyribonucleic acid).

However, the conventional techniques mentioned above require disrupting the bacterial cell of the PCC6803 strain for retrieving protein produced within the bacterial cell of the PCC6803 strain. Hence, the production of the protein is time-consuming. Since various materials exist within the bacterial cell, it may be necessary to remove these materials and purify the protein of interest. Hence, the protein yield is decreased. It is also necessary to provide a fresh bacterial strain for each protein production, which is time-consuming and increases production cost. Thus, the conventional techniques mentioned above still have a low level of efficiency of protein production using cyanobacterium. There is a demand for the development of technology with higher production efficiency.

Accordingly, the present inventor has found that protein produced within the bacterial cell of cyanobacterium is easily secreted to the outside of the bacterial cell by partially detaching the outer membrane which surrounds the cell wall of cyanobacterium from the cell wall. As a result, the protein secreted to the outside of the bacterial cell can be efficiently retrieved without disrupting the bacterial cell of the cyanobacterium. Furthermore, production efficiency is improved because the cyanobacterium can be continuously used even after protein retrieval.

Therefore, the present disclosure can provide a modified cyanobacterium having improved protein secretory productivity. Furthermore, by culturing the modified cyanobacterium according to the present disclosure, protein can be efficiently produced.

### (Outline of Present Disclosure)

An outline of an aspect of the present disclosure is as described below.

A modified cyanobacterium according to an aspect of the present disclosure is a modified cyanobacterium in which a function of a protein involved in binding between an outer membrane and a cell wall of cyanobacterium is suppressed or lost.

As a result, the binding (e.g., binding level and binding force) between the cell wall and the outer membrane is partially reduced in the modified cyanobacterium. This facilitates partially detaching the outer membrane from the cell wall. Hence, protein produced within the bacterial cell easily leaks out to the outside of the outer membrane, i.e., the outside of the bacterial cell. Thus, the modified cyanobacterium according to an aspect of the present disclosure can provide cyanobacterium having improved secretory productivity of protein. Furthermore, the modified cyanobacterium according to an aspect of the present disclosure eliminates the need of retrieving protein by disrupting the bacterial cell and can therefore be repeatedly used in protein production even after protein retrieval.

For example, in the modified cyanobacterium according to an aspect of the present disclosure, the protein involved in the binding between the outer membrane and the cell wall may be at least one of a surface layer homology (SLH) domain-containing outer membrane protein or a cell wall-pyruvic acid modifying enzyme.

As a result, in the modified cyanobacterium, for example, (i) a function of at least one of a SLH domain-containing outer membrane protein which binds to the cell wall and an enzyme that catalyzes reaction to modify a linked sugar chain on the surface of the cell wall with pyruvic acid (i.e., a cell wall-pyruvic acid modifying enzyme) is suppressed or lost, or (ii) the expression of at least one of the SLH domain-containing outer membrane protein or the cell wall-pyruvic acid modifying enzyme is suppressed. Hence, the binding (i.e., binding level and binding force) between the SLH domain of the SLH domain-containing outer membrane protein in the outer membrane and a covalently linked sugar chain on the surface of the cell wall is reduced. Accordingly, the outer membrane is easily detached from the cell wall at a site having the weakened binding between the outer membrane and the cell wall. Thus, according to the modified cyanobacterium according to an aspect of the present disclosure, protein produced within the bacterial cell easily leaks out to the outside of the bacterial cell because the outer membrane is easy to partially detach from the cell wall due to the weakened binding between the cell wall and the outer membrane.

For example, in the modified cyanobacterium according to an aspect of the present disclosure, the SLH domain-containing outer membrane protein may be: SIr1841 having an amino acid sequence represented by SEQ ID NO: 1; NIES970_09470 having an amino acid sequence represented by SEQ ID NO: 2; Anacy_3458 having an amino acid sequence represented by SEQ ID NO: 3; or a protein having an amino acid sequence that is at least 50 percent identical to the amino acid sequence of any one of the SIr1841, the NIES970_09470, and the Anacy_3458.

As a result, in the modified cyanobacterium, for example, (i) the function of the SLH domain-containing outer membrane protein represented by any one of SEQ ID NOs: 1 to 3 or a protein having an amino acid sequence that is at least 50 percent identical to the amino acid sequence of any one of these SLH domain-containing outer membrane proteins is suppressed or lost, or (ii) the expression of the SLH domain-containing outer membrane protein represented by any one of SEQ ID NOs: 1 to 3 or a protein having an amino acid sequence that is at least 50 percent identical to the amino acid sequence of any one of these SLH domain-containing outer membrane proteins is suppressed. Hence, in the modified cyanobacterium, (i) the function of the SLH domain-containing outer membrane protein or a protein functionally equivalent to the SLH domain-containing outer membrane protein in the outer membrane is suppressed or lost, or (ii) the expression level of the SLH domain-containing outer membrane protein or a protein functionally equivalent to the SLH domain-containing outer membrane protein in the outer membrane is decreased. Therefore, according to the modified cyanobacterium according to an aspect of the present disclosure, the binding level and binding force with which a binding domain (e.g., the SLH domain) for binding the outer membrane with the cell wall binds to the cell wall are reduced. This facilitates partially detaching the outer membrane from the cell wall.

For example, in the modified cyanobacterium according to an aspect of the present disclosure, the cell wall-pyruvic acid modifying enzyme may be: SIr0688 having an amino acid sequence represented by SEQ ID NO: 4; Synpcc7942_1529 having an amino acid sequence represented by SEQ ID NO: 5; Anacy_1623 having an amino acid sequence represented by SEQ ID NO: 6; or a protein having an amino acid sequence that is at least 50 percent identical to the amino acid sequence of any one of the SIr0688, the Synpcc7942_1529, and the Anacy_1623.

As a result, in the modified cyanobacterium, for example, (i) the function of the cell wall-pyruvic acid modifying enzyme represented by any one of SEQ ID NOs: 4 to 6 or a protein having an amino acid sequence that is at least 50 percent identical to the amino acid sequence of any one of these cell wall-pyruvic acid modifying enzymes is suppressed or lost, or (ii) the expression of the cell wall-pyruvic acid modifying enzyme represented by any one of SEQ ID NOs: 4 to 6 or a protein having an amino acid sequence that is at least 50 percent identical to the amino acid sequence of any one of these cell wall-pyruvic acid modifying enzymes is suppressed. Hence, in the modified cyanobacterium, (i) the function of the cell wall-pyruvic acid modifying enzyme or a protein functionally equivalent to the enzyme is suppressed or lost, or ii) the expression level of the cell wall-pyruvic acid modifying enzyme or a protein functionally equivalent to the enzyme is decreased. A covalently linked sugar chain on the surface of the cell wall is thereby less susceptible to modification with pyruvic acid, so that binding level and binding force of the sugar chain of the cell wall that binds to the SLH domain of the SLH domain-containing outer membrane protein in the outer membrane are reduced. Therefore, in the modified cyanobacterium according to an aspect of the present disclosure, a covalently linked sugar chain on the surface of the cell wall is less susceptible to modification with pyruvic acid, so that binding force between the cell wall and the outer membrane is weakened. This facilitates partially detaching the outer membrane from the cell wall.

For example, in the modified cyanobacterium according to an aspect of the present disclosure, a gene which causes expression of the protein involved in the binding between the outer membrane and the cell wall may be deleted or inactivated.

Accordingly, in the modified cyanobacterium, the expression of the protein involved in the binding between the cell wall and the outer membrane is suppressed, or the function of the protein is suppressed or lost. Therefore, the binding (i.e., binding level and binding force) between the cell wall and the outer membrane is partially reduced. As a result, in the modified cyanobacterium, the outer membrane is easy to partially detach from the cell wall, so that protein produced within the bacterial cell easily leaks out to the outside of the outer membrane, i.e., the outside of the bacterial cell. Thus, the modified cyanobacterium according to an aspect of the present disclosure has improved secretory productivity of protein. Furthermore, the modified cyanobacterium according to an aspect of the present disclosure eliminates the need of retrieving protein by disrupting the bacterial cell and can therefore be repeatedly used in protein production even after protein retrieval.

For example, in the modified cyanobacterium according to an aspect of the present disclosure, the gene which causes expression of the protein involved in the binding between the outer membrane and the cell wall may be at least one of a gene encoding an SLH domain-containing outer membrane protein or a gene encoding a cell wall-pyruvic acid modifying enzyme.

Accordingly, in the modified cyanobacterium, at least one of the gene encoding the SLH domain-containing outer membrane protein and the gene encoding the cell wall-pyruvic acid modifying enzyme is deleted or inactivated. Hence, in the modified cyanobacterium, for example, (i) the expression of at least one of the SLH domain-containing outer membrane protein or the cell wall-pyruvic acid modifying enzyme is suppressed, or (ii) the function of at least one of the SLH domain-containing outer membrane protein or the cell wall-pyruvic acid modifying enzyme is suppressed or lost. Hence, the binding (i.e., binding level and binding force) between the SLH domain of the SLH domain-containing outer membrane protein in the outer membrane and a covalently linked sugar chain on the surface of the cell wall is reduced. Accordingly, the outer membrane is easily detached from the cell wall at a site having the weakened binding between the outer membrane and the cell wall. Thus, according to the modified cyanobacterium according to an aspect of the present disclosure, protein produced within the bacterial cell easily leaks out to the outside of the bacterial cell because the outer membrane is easy to partially detach from the cell wall due to the weakened binding between the cell wall and the outer membrane.

For example, in the modified cyanobacterium according to an aspect of the present disclosure, the gene encoding the SLH domain-containing outer membrane protein may be: sIr1841 having a nucleotide sequence represented by SEQ ID NO: 7; nies970_09470 having a nucleotide sequence represented by SEQ ID NO: 8; anacy_3458 having a nucleotide sequence represented by SEQ ID NO: 9; or a gene having a nucleotide sequence that is at least 50 percent identical to the nucleotide sequence of any one of the sIr1841, the nies970_09470, and the anacy_3458.

As a result, in the modified cyanobacterium, the gene encoding the SLH domain-containing outer membrane protein, represented by any one of SEQ ID NOs: 7 to 9 or a gene having a nucleotide sequence that is at least 50 percent identical to the nucleotide sequence of any one of these genes is deleted or inactivated. Hence, in the modified cyanobacterium, (i) the expression of any one of the SLH domain-containing outer membrane proteins described above or a protein functionally equivalent to any one of these proteins is suppressed, or (i) the function of any one of the SLH domain-containing outer membrane proteins described above or a protein functionally equivalent to any one of these proteins is suppressed or lost. Thus, according to the modified cyanobacterium according to an aspect of the present disclosure, the binding level and binding force of a binding domain (e.g., the SLH domain) of the outer membrane that binds to the cell wall are reduced. This facilitates partially detaching the outer membrane from the cell wall.

For example, in the modified cyanobacterium according to an aspect of the present disclosure, the gene encoding the cell wall-pyruvic acid modifying enzyme may be: sIr0688 having a nucleotide sequence represented by SEQ ID NO: 10; synpcc7942_1529 having a nucleotide sequence represented by SEQ ID NO: 11; anacy_1623 having a nucleotide sequence represented by SEQ ID NO: 12; or a gene having a nucleotide sequence that is at least 50 percent identical to the nucleotide sequence of any one of the sIr0688, the synpcc7942_1529, and the anacy_1623.

As a result, in the modified cyanobacterium, the gene encoding the cell wall-pyruvic acid modifying enzyme, represented by any one of SEQ ID NOs: 10 to 12 or a gene having a nucleotide sequence that is at least 50 percent identical to the nucleotide sequence of any one of these enzyme-encoding genes is deleted or inactivated. Hence, in the modified cyanobacterium, (i) the expression of any one of the cell wall-pyruvic acid modifying enzymes described above or a protein functionally equivalent to any one of these enzymes is suppressed, or (ii) the function of any one of the cell wall-pyruvic acid modifying enzymes described above or a protein functionally equivalent to any one of these enzymes is suppressed or lost. A covalently linked sugar chain on the surface of the cell wall is thereby less susceptible to modification with pyruvic acid, so that binding level and binding force of the sugar chain of the cell wall that binds to the SLH domain of the SLH domain-containing outer membrane protein in the outer membrane are reduced. Thus, in the modified cyanobacterium according to an aspect of the present disclosure, a decreased amount of a sugar chain on cell wall that binds to the outer membrane is modified with pyruvic acid, so that binding force between the cell wall and the outer membrane is weakened. This facilitates partially detaching the outer membrane from the cell wall.

Furthermore, a modified cyanobacterium production method according to an aspect of the present disclosure includes: causing a function of a protein involved in binding between an outer membrane and a cell wall of cyanobacterium to be suppressed or lost.

As a result, the binding (e.g., binding level and binding force) between the cell wall and the outer membrane is partially reduced in the produced modified cyanobacterium. This facilitates partially detaching the outer membrane from the cell wall. Hence, in the modified cyanobacterium, protein produced within the bacterial cell easily leaks out to the outside of the outer membrane (i.e., the outside of the bacterial cell). Thus, the modified cyanobacterium production method according to an aspect of the present disclosure can provide cyanobacterium having improved secretory productivity of protein. Furthermore, the produced modified cyanobacterium eliminates the need of disrupting the bacterial cell for protein retrieval because protein produced within the bacterial cell easily leaks out to the outside of the bacterial cell. Thus, the modified cyanobacterium production method according to an aspect of the present disclosure can provide a modified cyanobacterium with high use efficiency that can be repeatedly used even after protein retrieval.

Furthermore, a protein production method according to an aspect of the present disclosure includes culturing any one of the above-described modified cyanobacterium.

As a result, the modified cyanobacterium enables protein that has leaked out to the outside of the bacterial cell to be retrieved and therefore eliminates the need of retrieving the bacterial cell from a culture solution and disrupting the bacterial cell for protein retrieval. While the modified cyanobacterium is cultured, the protein can be retrieved. Furthermore, the modified cyanobacterium can be repeatedly used even after protein retrieval and therefore eliminates the need of providing a fresh modified cyanobacterium for each protein production. Thus, the protein production method according to an aspect of the present disclosure can efficiently produce protein.

Hereinafter, embodiments will be described in detail with reference to the drawings.

It should be noted that each of the subsequently described embodiments shows a generic or specific example. Numerical values, materials, steps, the processing order of the steps indicated in the following embodiments are merely examples, and thus are not intended to limit the present disclosure. Furthermore, among the elements in the following embodiments, elements that are not described in the independent claims indicating the broadest concepts are described as optional elements.

Furthermore, the figures are not necessarily precise illustrations. In the figures, elements that are substantially the same are given the same reference signs, and overlapping description thereof may be omitted or simplified.

Moreover, in the following embodiments, numerical ranges include, not only the precise meanings, but also substantially equal ranges, such as, for example, a measured amount (for example, the number, the concentration, etc.) of a protein or a range thereof, etc.

In the present specification, both of a bacterial cell and a cell refer to one individual of cyanobacterium.

### EMBODIMENT

### 1. Definitions

In the present specification, the identity of a nucleotide sequence or an amino acid sequence is calculated with Basic Local Alignment Search Tool (BLAST) algorithm. Specifically, the identity is calculated by pairwise analysis with the BLAST program available in the website of the National Center for Biotechnology Information (NCBI) (https://blast.ncbi.nlm.nih.gov/Blast.cgi). Information on cyanobacterium genes and proteins encoded by these genes are published in, for example, the NCBI database mentioned above and Cyanobase (http://genome.microbedb.jp/cyanobase/). The amino acid sequence of the protein of interest and the nucleotide sequence of a gene encoding the protein can be obtained from these databases.

Cyanobacterium, also called blue-green alga or blue-green bacterium, is a group of prokaryote that collects light energy through chlorophyll and performs photosynthesis while generating oxygen through the splitting of water using the obtained energy. Cyanobacterium is highly diverse and includes, for example, unicellular species such as Synechocystis sp. PCC 6803 and filamentous species having multicellular filaments such as Anabaena sp. PCC 7120, in terms of cell shape. There are also thermophilic species such as Thermosynechococcus elongatus, marine species such as Synechococcus elongatus, and freshwater species such as Synechocystis, in terms of growth environment. Other examples thereof include many species having unique features, including species, such as Microcystis aeruginosa, which have a gas vesicle and produce toxin, and Geobacter violaceus which lacks thylakoid and has a light-harvesting antenna protein called phycobilisome in the plasma membrane.

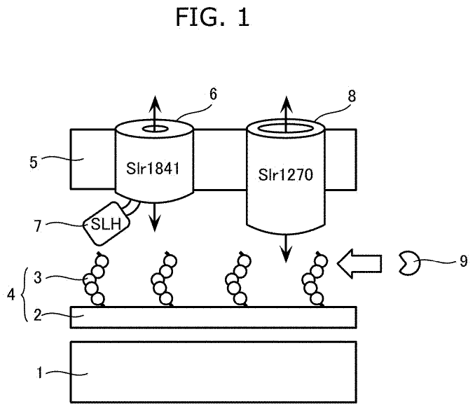

FIG. 1 is a diagram schematically illustrating a cell surface of a cyanobacterium. As illustrated in FIG. 1, the cell surface of cyanobacterium is constituted by a plasma membrane (also referred to as inner membrane 1), peptidoglycan 2, and outer membrane 5 which is a lipid membrane that forms the outermost layer of the cell, in order from the inside. Sugar chain 3 constituted by glucosamine and mannosamine, etc. is covalently linked to peptidoglycan 2, and pyruvic acid is bound with this covalently linked sugar chain 3 (NPL 5: Jurgens and Weckesser, 1986, J. Bacteriol., 168: 568-573). In the present specification, peptidoglycan 2 and covalently linked sugar chain 3 are collectively referred to as cell wall 4. The space between the plasma membrane (i.e., inner membrane 1) and outer membrane 5 is called periplasm where various enzymes involved in protein degradation or conformation formation, lipid or nucleic acid degradation, or uptake of extracellular nutrients, etc. are present.

A SLH domain-containing outer membrane protein (e.g., SIr1841 in the figure) has a C-terminal region embedded in a lipid membrane (also referred to as outer membrane 5) and N-terminal SLH domain 7 projecting from the lipid membrane, and is widely distributed in cyanobacterium and bacteria belonging to the class Negativicutes, a group of Gram-negative bacteria (NPL 6: Kojima et al., 2016, Biosci. Biotech. Biochem., 10: 1954-1959). The region embedded in the lipid membrane (i.e., outer membrane 5) forms a channel that allows hydrophilic materials to permeate the outer membrane, whereas SLH domain 7 has a function of binding to cell wall 4 (NPL 7: Kowata et al., 2017, J. Bacteriol., 199: e00371-17). The binding of SLH domain 7 to cell wall 4 requires modifying covalently linked sugar chain 3 on peptidoglycan 2 with pyruvic acid (Kojima et al., 2016, J. Biol. Chem., 291: 20198-20209). Examples of the gene encoding SLH domain-containing outer membrane protein 6 include sIr1841 and sIr1908 retained by Synechocystis sp. PCC 6803, and oprB retained by Anabaena sp. 90.

An enzyme that catalyzes the pyruvic acid modification reaction of covalently linked sugar chain 3 (hereinafter, referred to as cell wall-pyruvic acid modifying enzyme 9) in peptidoglycan 2 was identified in a Gram-positive bacterium Bacillus anthracis and designated as CsaB (NPL 8: Mesnage et al., 2000, EMBO J., 19: 4473-4484). Many species of cyanobacterium whose genomic nucleotide sequence is published retains a gene encoding a homologous protein having an amino acid sequence that has 30% or higher identity to the amino acid sequence of CsaB. Examples thereof include sIr0688 retained by Synechocystis sp. PCC 6803 and synpcc7502_03092 retained by Synechococcus sp. 7502.

In cyanobacterium, CO₂ fixed by photosynthesis is converted to various amino acids through multiple stages of enzymatic reaction. Protein is synthesized in the cytoplasm of cyanobacterium with these amino acids as starting materials. Such protein includes protein that functions in the cytoplasm and protein that is transported from the cytoplasm to the periplasm and functions in the periplasm. However, any case where protein is actively secreted to the outside of the cell has not been reported on cyanobacterium so far.

Cyanobacterium has high photosynthetic ability and therefore need not necessarily to take up organic substances as nutrients from the outside. Hence, cyanobacterium has only a very small amount of a channel protein, such as organic channel protein 8 (e.g., SIr1270) of FIG. 1, which permits permeation of organic substances, in outer membrane 5. For example, Synechocystis sp. PCC 6803 has only approximately 4% of organic channel protein 8 which permits permeation of organic substances based on the amount of total protein in outer membrane 5. On the other hand, outer membrane 5 of cyanobacterium is rich in an ion channel protein, such as SLH domain-containing outer membrane protein 6 (e.g., SIr1841) of FIG. 1, which permits selective permeation of only inorganic ions, for high-efficiency cellular uptake of inorganic ions necessary for growth. For example, in Synechocystis sp. PCC 6803, the ion channel protein which permits permeation of inorganic ions accounts for approximately 80% of the total protein of outer membrane 5.

Thus, cyanobacterium is considered to have the difficulty in actively secreting protein produced within the bacterial cell to the outside of the bacterial cell, due to very few channels which permit permeation of organic substances such as protein in outer membrane 5.

### 2. Modified Cyanobacterium

Subsequently, the modified cyanobacterium according to the present embodiment will be described with reference to FIG. 1.

In the modified cyanobacterium according to the present embodiment, a function of a protein involved in binding between outer membrane 5 and cell wall 4 (hereinafter, also referred to as a binding-related protein) of cyanobacterium is suppressed or lost. As a result, the binding (e.g., binding level and binding force) between outer membrane 5 and cell wall 4 is partially reduced in the modified cyanobacterium. This facilitates partially detaching outer membrane 5 from cell wall 4. Hence, the modified cyanobacterium has improved secretory productivity of protein that is produced within the bacterial cell and secreted to the outside of the bacterial cell. Furthermore, the modified cyanobacterium eliminates the need of retrieving protein by disrupting the bacterial cell and can therefore be repeatedly used in protein production even after protein retrieval. In the present specification, to make protein within the bacterial cell by the modified cyanobacterium is referred to as production, and to secrete the produced protein to the outside of the bacterial cell is referred to as secretory production.

The protein involved in binding between outer membrane 5 and cell wall 4 may be at least one of SLH domain-containing outer membrane protein 6 or cell wall-pyruvic acid modifying enzyme 9. In the present embodiment, in the modified cyanobacterium, for example, the function of at least one of SLH domain-containing outer membrane protein 6 or cell wall-pyruvic acid modifying enzyme 9 is suppressed or lost. For example, in the modified cyanobacterium, (i) the function of at least one of SLH domain-containing outer membrane protein 6 or cell wall-pyruvic acid modifying enzyme 9 may be suppressed or lost, or (i) at least one of the expression of SLH domain-containing outer membrane protein 6 which binds to cell wall 4 or an enzyme that catalyzes the pyruvic acid modification reaction of a linked sugar chain on the surface of cell wall 4 (i.e., cell wall-pyruvic acid modifying enzyme 9) may be suppressed. As a result, the binding (e.g., binding level and binding force) between SLH domain 7 of SLH domain-containing outer membrane protein 6 in outer membrane 5 and covalently linked sugar chain 3 on the surface of cell wall 4 is reduced. Hence, outer membrane 5 is easily detached from cell wall 4 at a site having the weakened binding therebetween. Owing to partial detachment of outer membrane 5 from cell wall 4, protein present in the cell, particularly, the periplasm, of the modified cyanobacterium easily leaks out to the outside of the cell (outside of outer membrane 5).

As mentioned above, cyanobacterium has high photosynthetic ability and therefore need not necessarily to take up organic substances as nutrients from the outside. Hence, light, air, water and trace inorganic substances suffice for cyanobacterium culture. Cyanobacterium takes up inorganic substances into the cell through the ion channel of outer membrane 5 and intracellularly produces protein. Various proteins exist, particularly, in the periplasm of the space between outer membrane 5 and cell wall 4. In the modified cyanobacterium according to the present embodiment, a function of a protein involved in binding between outer membrane 5 and cell wall 4 is suppressed or lost. This facilitates partially detaching outer membrane 5 from cell wall 4. A periplasmic protein leaks out into a culture solution from a site where outer membrane 5 is detached. As a result, the modified cyanobacterium has improved secretory productivity of protein that is produced within the bacterial cell and secreted to the outside of the bacterial cell.

Hereinafter, a cyanobacterium modified so as to partially detach outer membrane 5 from cell wall 4 by suppressing a function of at least one binding-related protein of SLH domain-containing outer membrane protein 6 and cell wall-pyruvic acid modifying enzyme 9 will be specifically described.

The type of the cyanobacterium before at least one of the expression of SLH domain-containing outer membrane protein 6 or the expression of cell wall-pyruvic acid modifying enzyme 9 is suppressed or lost (hereinafter, referred to as a parent cyanobacterium), which serves as the parent microbe of the modified cyanobacterium according to the present embodiment, is not particularly limited and may be any type of cyanobacterium. The parent cyanobacterium may be, for example, the genus Synechocystis, Synechococcus, Anabaena, or Thermosynechococcus, and may be Synechocystis sp. PCC 6803, Synechococcus sp. PCC 7942, or Thermosynechococcus elongatus BP-1 among them.

The amino acid sequences of SLH domain-containing outer membrane protein 6 and the enzyme that catalyzes the pyruvic acid modification reaction of the cell wall (i.e., cell wall-pyruvic acid modifying enzyme 9) in the parent cyanobacterium, the nucleotide sequences of genes encoding these binding-related proteins, and the positions of the genes on chromosomal DNA or a plasmid can be confirmed in the NCBI database and Cyanobase mentioned above.

SLH domain-containing outer membrane protein 6 or cell wall-pyruvic acid modifying enzyme 9, the function of which is suppressed or lost in the modified cyanobacterium according to the present embodiment may be from any parent cyanobacterium and is not limited by the location where a gene encoding it resides (e.g., on chromosomal DNA or on a plasmid) as long as the parent cyanobacterium carries it.

SLH domain-containing outer membrane protein 6 may be, for example, SIr1841, SIr1908, or SIr0042 when the parent cyanobacterium is the genus Synechocystis, may be NIES970_09470, etc. when the parent cyanobacterium is the genus Synechococcus, may be Anacy_5815 or Anacy_3458, etc. when the parent cyanobacterium is the genus Anabaena, may be A0A0F6U6F8_MICAE, etc. when the parent cyanobacterium is the genus Microcystis, may be A0A3B8XX12_9CYAN, etc. when the parent cyanobacterium is the genus Cyanothece, may be A0A1Q8ZE23_9CYAN, etc, when the parent cyanobacterium is the genus Leptolyngbya, includes A0A1Z4R6U0_9CYAN when the parent cyanobacterium is the genus Calothrix, may be A0A1C0VG86_9NOSO, etc. when the parent cyanobacterium is the genus Nostoc, may be B1WRN6_CROS5, etc. when the parent cyanobacterium is the genus Crocosphaera, and may be K9TAE4_9CYAN, etc. when the parent cyanobacterium is the genus Pleurocapsa.

More specifically, SLH domain-containing outer membrane protein 6 may be, for example, SIr1841 (SEQ ID NO: 1) of Synechocystis sp. PCC 6803, NIES970_09470 (SEQ ID NO: 2) of Synechococcus sp. NIES-970, or Anacy_3458 (SEQ ID NO: 3) of Anabaena cylindrica PCC 7122. Alternatively, a protein having an amino acid sequence that is at least 50 percent identical to the amino acid sequence of any one of these SLH domain-containing outer membrane proteins 6 may be used.

As a result, in the modified cyanobacterium, for example, (i) the function of SLH domain-containing outer membrane protein 6 represented by any one of SEQ ID NOs: 1 to 3 or a protein having an amino acid sequence that is at least 50 percent identical to the amino acid sequence of any one of these SLH domain-containing outer membrane proteins 6 may be suppressed or lost, or (ii) the expression of SLH domain-containing outer membrane protein 6 represented by any one of SEQ ID NOs: 1 to 3 or a protein having an amino acid sequence that is at least 50 percent identical to the amino acid sequence of any one of these SLH domain-containing outer membrane proteins 6 may be suppressed. Hence, in the modified cyanobacterium, (i) the function of SLH domain-containing outer membrane protein 6 or a protein functionally equivalent to SLH domain-containing outer membrane protein 6 in outer membrane 5 is suppressed or lost, or (ii) the expression level of SLH domain-containing outer membrane protein 6 or a protein functionally equivalent to SLH domain-containing outer membrane protein 6 in outer membrane 5 is decreased. Therefore, in the modified cyanobacterium according to the present embodiment, the binding level and binding force with which a binding domain (e.g., SLH domain 7) for binding outer membrane 5 with cell wall 4 binds to cell wall 4 are reduced. This facilitates partially detaching outer membrane 5 from cell wall 4.

In general, a protein having an amino acid sequence that is at least 30 percent identical to the amino acid sequence of a protein has high conformational homology to the protein and therefore, is reportedly likely to be functionally equivalent to the protein. Hence, SLH domain-containing outer membrane protein 6, the function of which is suppressed or lost may be, for example, a protein or a polypeptide which has an amino acid sequence that has 40% or higher, preferably 50% or higher, more preferably 60% or higher, further preferably 70% or higher, still further preferably 80% or higher, even further preferably 90% or higher identity to the amino acid sequence of SLH domain-containing outer membrane protein 6 represented by any one of SEQ ID NOs: 1 to 3, and which has a function of binding to covalently linked sugar chain 3 of cell wall 4.

Cell wall-pyruvic acid modifying enzyme 9 may be, for example, SIr0688 when the parent cyanobacterium is the genus Synechocystis, may be Syn7502_03092 or Synpcc7942_1529, etc. when the parent cyanobacterium is the genus Synechococcus, may be ANA_C20348 or Anacy_1623, etc. when the parent cyanobacterium is the genus Anabaena, may be CsaB (NCBI accession ID: TRU80220), etc. when the parent cyanobacterium is the genus Microcystis, may be CsaB (NCBI accession ID: WP_107667006.1), etc. when the parent cyanobacterium is the genus Cyanothece, may be CsaB (NCBI accession ID: WP_026079530.1), etc. when the parent cyanobacterium is the genus Spirulina, may be CsaB (NCBI accession ID: WP_096658142.1), etc. when the parent cyanobacterium is the genus Calothrix, may be CsaB (NCBI accession ID: WP_099068528.1), etc. when the parent cyanobacterium is the genus Nostoc, may be CsaB (NCBI accession ID: WP_012361697.1), etc. when the parent cyanobacterium is the genus Crocosphaera, and may be CsaB (NCBI accession ID: WP_036798735), etc. when the parent cyanobacterium is the genus Pleurocapsa.

More specifically, cell wall-pyruvic acid modifying enzyme 9 may be, for example, SIr0688 (SEQ ID NO: 4) of Synechocystis sp. PCC 6803, Synpcc7942_1529 (SEQ ID NO: 5) of Synechococcus sp. PCC 7942, or Anacy_1623 (SEQ ID NO: 6) of Anabaena cylindrica PCC 7122. Alternatively, a protein having an amino acid sequence that is at least 50 percent identical to the amino acid sequence of any one of these cell wall-pyruvic acid modifying enzymes 9 may be used.

As a result, in the modified cyanobacterium, for example, (i) the function of cell wall-pyruvic acid modifying enzyme 9 represented by any one of SEQ ID NOs: 4 to 6 or a protein having an amino acid sequence that is at least 50 percent identical to the amino acid sequence of any one of these cell wall-pyruvic acid modifying enzymes 9 is suppressed or lost, or (ii) the expression of cell wall-pyruvic acid modifying enzyme 9 represented by any one of SEQ ID NOs: 4 to 6 or a protein having an amino acid sequence that is at least 50 percent identical to the amino acid sequence of any one of these cell wall-pyruvic acid modifying enzymes 9 is suppressed. Hence, in the modified cyanobacterium, (i) the function of cell wall-pyruvic acid modifying enzyme 9 or a protein functionally equivalent to the enzyme is suppressed or lost, or (ii) the expression level of cell wall-pyruvic acid modifying enzyme 9 or a protein functionally equivalent to the enzyme is decreased. Covalently linked sugar chain 3 on the surface of cell wall 4 is thereby less susceptible to modification with pyruvic acid, so that binding level and binding force of sugar chain 3 of cell wall 4 that binds to SLH domain 7 of SLH domain-containing outer membrane protein 6 in outer membrane 5 are reduced. Therefore, in the modified cyanobacterium according to the present embodiment, covalently linked sugar chain 3 on the surface of cell wall 4 is less susceptible to modification with pyruvic acid, so that binding force between cell wall 4 and outer membrane 5 is weakened. This facilitates partially detaching outer membrane 5 from cell wall 4.

As mentioned above, a protein having an amino acid sequence that is at least 30 percent identical to the amino acid sequence of a protein is reportedly likely to be functionally equivalent to the protein. Hence, cell wall-pyruvic acid modifying enzyme 9, the function of which is suppressed or lost may be, for example, a protein or a polypeptide which has an amino acid sequence that has 40% or higher, preferably 50% or higher, more preferably 60% or higher, further preferably 70% or higher, still further preferably 80% or higher, even further preferably 90%/Y or higher identity to the amino acid sequence of cell wall-pyruvic acid modifying enzyme 9 represented by any one of SEQ ID NOs: 4 to 6, and which has a function of catalyzing reaction to modify covalently linked sugar chain 3 on peptidoglycan 2 of cell wall 4 with pyruvic acid.

In the present specification, the phrase “a function of SLH domain-containing outer membrane protein 6 is suppressed or lost” means that the ability of the protein to bind to cell wall 4 is suppressed or lost, the transport of the protein to outer membrane 5 is suppressed or lost, or the ability of the protein to be embedded so as to function in outer membrane 5 is suppressed or lost.

The phrase “a function of cell wall-pyruvic acid modifying enzyme 9 is suppressed or lost” means that the function of modifying covalently linked sugar chain 3 of cell wall 4 with pyruvic acid by the protein is suppressed or lost.

An approach for suppressing or losing the functions of these proteins is not particularly limited as long as the approach is usually used for suppressing or losing protein functions. The approach may involve, for example, deleting or inactivating a gene encoding SLH domain-containing outer membrane protein 6 and a gene encoding cell wall-pyruvic acid modifying enzyme 9, inhibiting the transcription of these genes, inhibiting the translation of transcripts of these genes, or administrating inhibitors which specifically inhibit these proteins.

In the present embodiment, in the modified cyanobacterium, a gene which causes expression of the protein involved in binding between outer membrane 5 and cell wall 4 may be deleted or inactivated. As a result, in the modified cyanobacterium, the expression of the protein involved in the binding between cell wall 4 and outer membrane 5 is suppressed, or the function of the protein is suppressed or lost. Therefore, the binding (i.e., binding level and binding force) between cell wall 4 and outer membrane 5 is partially reduced. As a result, in the modified cyanobacterium, outer membrane 5 is easy to partially detach from cell wall 4, so that protein produced within the bacterial cell easily leaks out to the outside of outer membrane 5, i.e., the outside of the bacterial cell. Hence, the modified cyanobacterium according to the present embodiment has improved secretory productivity of protein that is produced within the bacterial cell and secreted to the outside of the bacterial cell. Furthermore, the modified cyanobacterium eliminates the need of retrieving protein by disrupting the bacterial cell and can therefore be repeatedly used in protein production even after protein retrieval.

The gene which causes expression of the protein involved in binding between outer membrane 5 and cell wall 4 may be, for example, at least one of a gene encoding SLH domain-containing outer membrane protein 6 or a gene encoding cell wall-pyruvic acid modifying enzyme 9. In the modified cyanobacterium, at least one of the gene encoding SLH domain-containing outer membrane protein 6 or the gene encoding cell wall-pyruvic acid modifying enzyme 9 is deleted or inactivated. Hence, in the modified cyanobacterium, for example, (i) the expression of at least one of SLH domain-containing outer membrane protein 6 or cell wall-pyruvic acid modifying enzyme 9 is suppressed, or (ii) the function of at least one of SLH domain-containing outer membrane protein 6 or cell wall-pyruvic acid modifying enzyme 9 is suppressed or lost. Hence, the binding (i.e., binding level and binding force) between SLH domain 7 of SLH domain-containing outer membrane protein 6 in outer membrane 5 and covalently linked sugar chain 3 on the surface of cell wall 4 is reduced. As a result, outer membrane 5 is easily detached from cell wall 4 at a site having the weakened binding between outer membrane 5 and cell wall 4. Thus, according to the modified cyanobacterium according to the present embodiment, protein produced within the bacterial cell easily leaks out to the outside of the bacterial cell because outer membrane 5 is easy to partially detach from cell wall 4 by the weakened binding between outer membrane 5 and cell wall 4.

In the present embodiment, for example, the transcription of at least one of the gene encoding SLH domain-containing outer membrane protein 6 or the gene encoding cell wall-pyruvic acid modifying enzyme 9 may be suppressed in order to suppress or lose the function of at least one of SLH domain-containing outer membrane protein 6 or cell wall-pyruvic acid modifying enzyme 9 in cyanobacterium.

The gene encoding SLH domain-containing outer membrane protein 6 may be, for example, sIr1841, sIr1908, or sIr0042 when the parent cyanobacterium is the genus Synechocystis, may be nies970_09470, etc. in the case of the genus Synechococcus, may be anacy_5815 or anacy_3458, etc. when the parent cyanobacterium is the genus Anabaena, may be A0A0F6U6F8 MICAE, etc. when the parent cyanobacterium is the genus Microcystis, may be A0A3B8XX12_9CYAN, etc. when the parent cyanobacterium is the genus Cyanothece, may be A0A1Q8ZE23_9CYAN, etc. when the parent cyanobacterium is the genus Leptolyngbya, may be A0A1Z4R6U0_9CYAN, etc. when the parent cyanobacterium is the genus Calothrix, may be A0A1C0VG86_9NOSO, etc. when the parent cyanobacterium is the genus Nostoc, may be B1WRN6_CROS5, etc. when the parent cyanobacterium is the genus Crocosphaera, and may be K9TAE4_9CYAN, etc. when the parent cyanobacterium is the genus Pleurocapsa. The nucleotide sequences of these genes can be obtained from the NCBI database or Cyanobase mentioned above.

More specifically, the gene encoding SLH domain-containing outer membrane protein 6 may be sIr1841 (SEQ ID NO: 7) of Synechocystis sp. PCC 6803, nies970_09470 (SEQ ID NO: 8) of Synechococcus sp. NIES-970, anacy_3458 (SEQ ID NO: 9) of Anabaena cylindrica PCC 7122, or a gene having a nucleotide sequence that is at least 50 percent identical to the nucleotide sequence of any one of these genes.

As a result, in the modified cyanobacterium, the gene encoding SLH domain-containing outer membrane protein 6, represented by any one of SEQ ID NOs: 7 to 9 or a gene having a nucleotide sequence that is at least 50 percent identical to the nucleotide sequence of any one of these genes is deleted or inactivated. Hence, in the modified cyanobacterium, (i) the expression of any one of SLH domain-containing outer membrane proteins 6 described above or a protein functionally equivalent to any one of these proteins is suppressed, or (ii) the function of any one of SLH domain-containing outer membrane proteins 6 described above or a protein functionally equivalent to any one of these proteins is suppressed or lost. Therefore, in the modified cyanobacterium according to the present embodiment, the binding level and binding force of a cell wall 4 binding domain (e.g., SLH domain 7) of outer membrane 5 that binds to cell wall 4 are reduced. This facilitates partially detaching outer membrane 5 from cell wall 4.

As mentioned above, a protein having an amino acid sequence that is at least 30 percent identical to the amino acid sequence of a protein is reportedly likely to be functionally equivalent to the protein. Hence, a gene having a nucleotide sequence that is at least 30 percent identical to the nucleotide sequence of a gene encoding a protein is considered likely to cause expression of a protein functionally equivalent to the protein. Hence, the gene encoding SLH domain-containing outer membrane protein 6, the function of which is suppressed or lost may be, for example, a gene which has a nucleotide sequence that has 40% or higher, preferably 50% or higher, more preferably 60% or higher, further preferably 70% or higher, still further preferably 80% or higher, even further preferably 90% or higher identity to the nucleotide sequence of the gene encoding SLH domain-containing outer membrane protein 6 represented by any one of SEQ ID NOs: 7 to 9, and which encodes a protein or a polypeptide having a function of binding to covalently linked sugar chain 3 of cell wall 4.

The gene encoding cell wall-pyruvic acid modifying enzyme 9 may be, for example, sIr0688 when the parent cyanobacterium is the genus Synechocystis, may be syn7502_03092 or synpcc7942_1529, etc. when the parent cyanobacterium is the genus Synechococcus, may be ana_C20348 or anacy_1623, etc. when the parent cyanobacterium is the genus Anabaena, may be csaB (NCBI accession ID: TRU80220), etc. when the parent cyanobacterium is the genus Microcystis, may be csaB (NCBI accession ID: WP_107667006.1), etc. when the parent cyanobacterium is the genus Cyanothece, may be csaB (NCBI accession ID:WP_026079530.1), etc. when the parent cyanobacterium is the genus Spirulina, may be csaB (NCBI accession ID:WIP_096658142.1), etc. when the parent cyanobacterium is the genus Calothrix, may be csaB (NCBI accession ID:WP_099068528.1), etc. when the parent cyanobacterium is the genus Nostoc, may be csaB (NCBI accession ID: WP_012361697.1), etc. when the parent cyanobacterium is the genus Crocosphaera, and may be csaB (NCBI accession ID: WP_036798735), etc. when the parent cyanobacterium is the genus Pleurocapsa. The nucleotide sequences of these genes can be obtained from the NCBI database or Cyanobase mentioned above.

More specifically, the gene encoding cell wall-pyruvic acid modifying enzyme 9 may be sIr0688 (SEQ ID NO: 10) of Synechocystis sp. PCC 6803, synpcc7942_1529 (SEQ ID NO: 11) of Synechococcus sp. PCC 7942, or anacy_1623 (SEQ ID NO: 12) of Anabaena cylindrica PCC 7122. Alternatively, a gene having a nucleotide sequence that is at least 50 percent identical to the nucleotide sequence of any one of these genes may be used.

As a result, in the modified cyanobacterium, the gene encoding cell wall-pyruvic acid modifying enzyme 9, represented by any one of SEQ ID NOs: 10 to 12 or a gene having a nucleotide sequence that is at least 50 percent identical to the nucleotide sequence of any one of these enzyme-encoding genes is deleted or inactivated. Hence, in the modified cyanobacterium, (i) the expression of any one of cell wall-pyruvic acid modifying enzymes 9 described above or a protein functionally equivalent to any one of these enzymes is suppressed, or (ii) the function of any one of cell wall-pyruvic acid modifying enzymes 9 described above or a protein functionally equivalent to any one of these enzymes is suppressed or lost. Covalently linked sugar chain 3 on the surface of cell wall 4 is thereby less susceptible to modification with pyruvic acid, so that binding level and binding force of sugar chain 3 of cell wall 4 that binds to SLH domain 7 of SLH domain-containing outer membrane protein 6 in outer membrane 5 are reduced. Thus, in the modified cyanobacterium according to the present embodiment, a decreased amount of sugar chain 3 on cell wall 4 that binds to outer membrane 5 is modified with pyruvic acid, so that binding force between cell wall 4 and outer membrane 5 is weakened. This facilitates partially detaching outer membrane 5 from cell wall 4.

As mentioned above, a gene having a nucleotide sequence that is at least 30 percent identical to the nucleotide sequence of a gene encoding a protein is considered likely to cause expression of a protein functionally equivalent to the protein. Hence, the gene encoding cell wall-pyruvic acid modifying enzyme 9, the function of which is suppressed or lost may be, for example, a gene which has a nucleotide sequence that has 40% or higher, preferably 50% or higher, more preferably 60% or higher, further preferably 70% or higher, still further preferably 80% or higher, even further preferably 90% or higher identity to the nucleotide sequence of the gene encoding cell wall-pyruvic acid modifying enzyme 9 represented by any one of SEQ ID NOs: 10 to 12, and which encodes a protein or a polypeptide having a function of catalyzing reaction to modify covalently linked sugar chain 3 on peptidoglycan 2 of cell wall 4 with pyruvic acid.

### 3. Modified Cyanobacterium Production Method

Subsequently, the modified cyanobacterium production method according to the present embodiment will be described. The modified cyanobacterium production method comprises causing a function of a protein involved in binding between outer membrane 5 and cell wall 4 of cyanobacterium to be suppressed or lost.

In the present embodiment, in the (i), the protein involved in binding between outer membrane 5 and cell wall 4 may be, for example, at least one of SLH domain-containing outer membrane protein 6 or cell wall-pyruvic acid modifying enzyme 9.

An approach for suppressing or losing the function of the protein is not particularly limited and may involve, for example, deleting or inactivating a gene encoding SLH domain-containing outer membrane protein 6 and a gene encoding cell wall-pyruvic acid modifying enzyme 9, inhibiting the transcription of these genes, inhibiting the translation of transcripts of these genes, or administrating inhibitors which specifically inhibit these proteins.

An approach for deleting or inactivating the gene may be, for example, the mutagenesis of one or more bases on the nucleotide sequence of the gene, the substitution of the nucleotide sequence by another nucleotide sequence, the insertion of another nucleotide sequence thereto, or the partial or complete deletion of the nucleotide sequence of the gene.

An approach for inhibiting the transcription of the gene may be, for example, the mutagenesis of a promoter region of the gene, the inactivation of the promoter by substitution by another nucleotide sequence or insertion of another nucleotide sequence, or CRISPR interference (NPL 9: Yao et al., ACS Synth. Biol., 2016, 5: 207-212). A specific approach for the mutagenesis or the substitution by or insertion of a nucleotide sequence may be, for example, ultraviolet irradiation, site-directed mutagenesis, or homologous recombination.

An approach for inhibiting the translation of a transcript of the gene may be, for example, RNA (ribonucleic acid) interference.

The function of the protein involved in binding between outer membrane 5 and cell wall 4 of cyanobacterium may be suppressed or lost to produce the modified cyanobacterium by use of any one of the above approaches.

As a result, the binding (e.g., binding level and binding force) between cell wall 4 and outer membrane 5 is partially reduced in the modified cyanobacterium produced by the production method according to the present embodiment. This facilitates partially detaching outer membrane 5 from cell wall 4. Hence, in the modified cyanobacterium, protein produced within the bacterial cell easily leaks out to the outside of outer membrane 5 (i.e., the outside of the bacterial cell). Thus, the modified cyanobacterium production method according to the present embodiment can provide a modified cyanobacterium having improved secretory productivity of protein.

The modified cyanobacterium produced by the production method according to the present embodiment eliminates the need of disrupting the bacterial cell for protein retrieval because protein produced within the bacterial cell easily leaks out to the outside of the bacterial cell. For example, the modified cyanobacterium can be cultured under appropriate conditions, and subsequently, protein secreted into the culture solution can be retrieved. Therefore, while the modified cyanobacterium is cultured, the protein in the culture solution may be retrieved. Hence, use of the modified cyanobacterium obtained by this production method enables efficient microbiological protein production to be carried out. Thus, the modified cyanobacterium production method according to the present embodiment can provide a modified cyanobacterium with high use efficiency that can be repeatedly used even after protein retrieval.

The modified cyanobacterium produced by the modified cyanobacterium production method according to the present embodiment secretes, for example, a protein group such as peptidase or phosphatase typically supposed to be present in the periplasm, to the outside of the cell. In the present embodiment, the modified cyanobacterium may be allowed to produce the desired protein, for example, by modifying a gene encoding a protein supposed to be produced within the cell of cyanobacterium, such as a protein group present in the periplasm, and replacing it with a gene encoding another protein. Thus, the modified cyanobacterium production method according to the present embodiment can also provide a modified cyanobacterium capable of conveniently and efficiently producing the desired protein.

### 4. Protein Production Method

Subsequently, the protein production method according to the present embodiment will be described. The protein production method according to the present embodiment comprises culturing the modified cyanobacterium described above.

Cyanobacterium culture can generally be carried out on the basis of liquid culture or a modified method thereof using a BG-11 medium (see Table 2). Hence, the culture of the modified cyanobacterium may be similarly carried out. The culture period of the cyanobacterium for protein production can be a period during which protein accumulates with a high concentration under conditions where the bacterial cell has proliferated sufficiently, and may be, for example, 1 to 3 days or may be 4 to 7 days. A culture method may be, for example, aeration and agitation culture or shake culture.

The modified cyanobacterium thus cultured under the conditions described above produces protein within the bacterial cell and secretes the protein into the culture solution. In the case of retrieving the protein secreted in the culture solution, insoluble materials such as the cell (i.e., the bacterial cell) may be removed from the culture solution by the filtration or centrifugation, etc. of the culture solution to retrieve a culture supernatant. The protein production method according to the present embodiment eliminates the need of disrupting the cell for protein retrieval because protein is secreted to the outside of the cell of the modified cyanobacterium. Hence, the modified cyanobacterium remaining after protein retrieval can be repeatedly used in protein production.

The method for retrieving the protein secreted into the culture solution is not limited to the example described above. While the modified cyanobacterium is cultured, the protein in the culture solution may be retrieved. For example, a protein-permeable membrane may be used to retrieve protein that has passed through the permeable membrane. In this case, a useful microbe such as lactic acid bacterium may be cultured by using, as a nutrient source, the protein that has passed through the permeable membrane. Thus, treatment to remove the bacterial cell of the modified cyanobacterium from a culture solution is unnecessary because, while the modified cyanobacterium is cultured, protein in the culture solution can be retrieved. Hence, protein can be produced more conveniently and efficiently.

Damage and stress on the modified cyanobacterium can be reduced because bacterial cell retrieval treatment from a culture solution and bacterial cell disruption treatment are unnecessary. Hence, the secretory productivity of protein is less likely to be reduced in the modified cyanobacterium, and the modified cyanobacterium can be used for a longer time.

Thus, the protein production method using the modified cyanobacterium according to the present embodiment can conveniently and efficiently produce an enzyme for food ingredient starting material or compound production, a diagnostic or therapeutic enzyme in the medical field, or an enzyme for feed in the agricultural, fishery, and livestock fields.

### WORKING EXAMPLES

Hereinafter, the modified cyanobacterium, the modified cyanobacterium production method, and the protein production method of the present disclosure will be specifically described with reference to working examples. However, the present disclosure is not limited by the following working examples by any means.

In the following working examples, two types of modified cyanobacteria were produced by suppressing the expression of sIr1841 gene encoding a SLH domain-containing outer membrane protein (Example 1) and suppressing the expression of sIr0688 gene encoding a cell wall-pyruvic acid modifying enzyme (Example 2) as methods for partially detaching the outer membrane of cyanobacterium from the cell wall. Then, the measurement of secretory productivity of protein and the identification of the secreted protein were performed as to these modified cyanobacteria. The cyanobacterium species used in the present working examples is Synechocystis sp. PCC 6803 (hereinafter, simply referred to as “cyanobacterium”).

### Working Example 1

In Example 1, a modified cyanobacterium was produced in which the expression of sIr1841 gene encoding a SLH domain-containing outer membrane protein was suppressed.

(1) Construction of modified cyanobacterium strain in which expression of sIr1841 gene was suppressed

The gene expression suppression method used was CRISPR (Clustered Regularly Interspaced Short Palindromic Repeat) interference. In this method, the expression of the sIr1841 gene can be suppressed by introducing a gene encoding dCas9 protein (hereinafter, referred to as dCas9 gene) and sIr1841_sgRNA (single-guide ribonucleic acid) gene to the chromosomal DNA of cyanobacterium.

The mechanism of the suppression of gene expression by this method is as follows.

First, nuclease activity-deficient Cas9 protein (dCas9) and sgRNA (sIr1841_sgRNA) complementarily binding to the nucleotide sequence of the sIr1841 gene forms a complex.

Next, this complex recognizes the sIr1841 gene on the chromosomal DNA of cyanobacterium and specifically binds to the sIr1841 gene. This binding, which serves as steric hindrance, inhibits the transcription of the sIr1841 gene. As a result, the expression of the sIr1841 gene in the cyanobacterium is suppressed.

Hereinafter, a method for introducing each of the two genes described above to the chromosomal DNA of cyanobacteriurn will be specifically described.

### (1-1) Introduction of dCas9 Gene

The dCas9 gene, operator gene for the expression control of the dCas9 gene, and spectinomycin resistance marker gene serving as an indicator for gene introduction were amplified by PCR (polymerase chain reaction) with the chromosomal DNA of a Synechocystis LY07 strain (hereinafter, also referred to as an LY07 strain) (see NPL 9) as a template using the primers psbA1-Fw (SEQ ID NO: 13) and psbA1-Rv (SEQ ID NO: 14) described in Table 1. In the LY07 strain, these three genes are inserted in a linked state in psbA1 gene on the chromosomal DNA and can therefore be amplified as one DNA fragment by PCR. Here, the obtained DNA fragment is referred to as a “psbA1::dCas9 cassette”. The psbA1::dCas9 cassette was inserted to a pUC19 plasmid by use of In-Fusion PCR Cloning® to obtain a pUC19-dCas9 plasmid.

TABLE 1

| | | SEQ |
|---|---|---|
| | | ID |
| Primer name | Nucleotide sequence | NO |
| psbA1-Fw | 5′-CAGTGAATTCGAGCTCGGTA | 13 |
| | TATAGCGTTGCAGTCCCTGG-3′ | |
| psbA1-Rv | 5′-GATTACGCCAAGGTTGCATG | 14 |
| | ACCGCGGTCACTTCATAACC-3′ | |
| slr2030-Fw | 5′-CAGTGAATTCGAGCTCGGTA | 15 |
| | ATAACCGTTGTCCCTTTTGTTTC | |
| | ATCG-3′ | |
| sgRNA_ | 5′-TGTTAGTGAGGCCTGCTGTT | 16 |
| slr1841-Rv | AGGTCGCAGTATCTCTATGACTG | |
| | AT-3′ | |
| sgRNA_ | 5′ACAGCAGGCTCAGTAACAGTT | 17 |
| slr1841-Fw | TTAGAGCTAGAAATAGCAAGTTA | |
| | AAATAA-3′ | |
| slr2031-Rv | 5′-GATTACGCCAAGCTTGCATG | 18 |
| | GGGAAGAATCTGAATCTGGGCAT | |
| | C-3′ | |
| sgRNA_ | 5′TTTTAGTCTGTTTGCTGGATA | 19 |
| slr0688-Rv | GCTGCGAGTATCTCTATCACTGA | |
| | T-3′ | |
| sgRNA_ | 5′-TGCAGCAAACAGACTAAAAG | 20 |
| slr0688-Fw | TTTTAGAGCTAGAAATAGCAAGT | |
| | TAAAATAA-3′ | |

1 μg of the obtained pUC19-dCas9 plasmid and a cyanobacterium culture solution (bacterial cell concentration OD730=approximately 0.5) were mixed, and the pUC19-dCas9 plasmid was introduced into the cyanobacterium cells by spontaneous transformation. The transformed cells were selected by growth on a BG-11 agar medium containing 20 μg/mL spectinomycin. In the selected cells, homologous recombination occurred between the psbA1 gene on the chromosomal DNA and the upstream and downstream fragment regions of psbA1 on the pUC19-dCas9 plasmid. In this way, a Synechocystis dCas9 strain having the insert of the dCas9 cassette in the psbA1 gene region was obtained. The composition of the BG-11 medium used is as described in Table 2.

TABLE 2

| Component | Content (mg/L) |
|---|---|
| EDTA-Na | 1 |
| Ammonium Iron Citrate | 6 |
| NaNO₃ | 1500 |
| MgSO₄ | 75 |
| K₂HPO₄ | 39 |
| CaCl₂ | 28.6 |
| H₂BO₄ | 2.8 |
| MnCl₂ | 1.8 |
| ZnSO₄ | 0.2 |
| CuSO₄ | 0.08 |
| Na₂MoO₄ | 0.02 |
| Co (NO₃) ₂ | 0.005 |
| TES-KOH (pH 7.5) | 4580 |

### (1-2) Introduction of sIr1841_sgRNA Gene

In the CRISPR interference, sgRNA specifically binds to a target gene by introducing a sequence of approximately 20 bases complementary to the target sequence to a region called protospacer on the sgRNA gene. The protospacer sequence used in the present working examples is described in Table 3.

TABLE 3

| Protospaer | Nucleotide | SEQ ID |
|---|---|---|
| target gene | sequence | NO |
| slr1841 | 5′-ACAGCAGGGCTCACTAACA-3′ | 21 |
| slr0688 | 5′-TGCAGCAAACAGAGTAAAA-3′ | 22 |

In the Synechocystis LY07 strain, the sgRNA gene (except for the protospacer region) and kanamycin resistance marker gene are inserted in a linked state in sIr2030-sIr2031 genes on the chromosomal DNA. Thus, sgRNA that specifically recognizes sIr1841 (sIr1841_sgRNA) can be easily obtained by adding a protospacer sequence (SEQ ID NO: 21) complementary to the sIr1841 gene (SEQ ID NO: 7) to primers for use in amplifying the sgRNA gene by PCR.

First, two DNA fragments were amplified by PCR with the chromosomal DNA of the LY07 strain as a template using a set of the primers sIr2030-Fw (SEQ ID NO: 15) and sgRNA_sIr1841-Rv (SEQ ID NO: 16) and a set of the primers sgRNA_sIr1841-Fw (SEQ ID NO: 17) and sIr2031-Rv (SEQ ID NO: 18) descried in Table 1.

Subsequently, a DNA fragment (sIr2030-2031::sIr1841_sgRNA) having (i) the sIr2030 gene fragment, (ii) sIr1841_sgRNA, (iii) kanamycin resistance marker gene, and (iv) the sIr2031 gene fragment linked in order was obtained by PCR amplification with a mixed solution of the DNA fragments described above as a template using the primers sIr2030-Fw (SEQ ID NO: 15) and sIr2031-Rv (SEQ ID NO: 18) described in Table 1. The sIr2030-2031::sIr1841_sgRNA was inserted to a pUC19 plasmid by use of In-Fusion PCR Cloning® to obtain a pUC19-sIr1841_sgRNA plasmid.

The pUC19-sIr1841_sgRNA plasmid was introduced to the Synechocystis dCas9 strain in the same manner as in the (1-1), and the transformed cells were selected on a BG-11 agar medium containing 30 μg/mL kanamycin. In this way, a transformant Synechocystis dCas9 sIr1841_sgRNA strain having the insert of sIr1841_sgRNA in the sIr2030-sIr2031 gene on the chromosomal DNA (hereinafter, also referred to as a sIr1841-suppressed strain) was obtained.

### (1-3) Suppression of sIr1841 Gene

The promoter sequences of the dCas9 gene and the sIr1841sgRNA gene were designed such that expression was induced in the presence of anhydrotetracycline (aTc). In the present working examples, the expression of the sIr1841 gene was suppressed by adding aTc (final concentration: 1 μg/mL) into the medium.

### Working Example 2

In Example 2, a modified cyanobacterium in which the expression of sIr0688 gene encoding a cell wall-pyruvic acid modifying enzyme was suppressed was obtained by the following procedures.

### (2) Construction of Modified Cyanobacterium Strain in which Expression of sIr0688 Gene was Suppressed

sgRNA gene containing a protospacer sequence (SEQ ID NO: 22) complementary to the sIr0688 gene (SEQ ID NO: 4) was introduced to the Synechocystis dCas9 strain by the same procedures as in the (1-2) to obtain a Synechocystis dCas9 sIr0688_sgRNA strain. The same conditions as in the (1-2) were used except that a set of the primers sIr2030-Fw (SEQ ID NO: 15) and sgRNA_sIr0688-Rv (SEQ ID NO: 19) and a set of the primers sgRNA_sIr0688-Fw (SEQ ID NO: 20) and sIr2031-Rv (SEQ ID NO: 18) described in Table 1 were used; and a DNA fragment (sIr2030-2031::sIr0688_sgRNA) having (i) the sIr2030 gene fragment, (ii) sIr0688_sgRNA, (iii) kanamycin resistance marker gene, and (iv) the sIr2031 gene fragment linked in order was inserted to a pUC19 plasmid by use of In-Fusion PCR Cloning® to obtain a pUC19-sIr0688sgRNA plasmid.

Further, the expression of the sIr0688 gene was suppressed by the same procedures as in the (1-3).

### Comparative Example 1

In Comparative Example 1, the Synechocystis dCas9 strain was obtained by the same procedures as in (1-1) of Example 1.

Subsequently, the state of the cell surface was observed and a protein secretory productivity test was performed as to each of the bacterial strains obtained in Example 1, Example 2 and Comparative Example 1. Hereinafter, the details will be described.

### (3) Observation of State of Cell Surface of Bacterial Strain

The respective ultrathin sections of the modified cyanobacterium Synechocystis dCas9 sIr1841_sgRNA strain (i.e., a sIr1841-suppressed strain) obtained in Example 1, the modified cyanobacterium Synechocystis dCas9 sIr0688_sgRNA strain (hereinafter, also referred to as a sIr0688-suppressed strain) obtained in Example 2, and the modified cyanobacterium Synechocystis dCas9 strain (hereinafter, referred to as a control strain) obtained in Comparative Example 1 were prepared, and the state of the cell surface (in other words, an outer membrane structure) was observed under an electron microscope.

### (3-1) Culture of Bacterial Strain

The sIr1841-suppressed strain of Example 1 was inoculated at initial bacterial cell concentration OD730=0.05 to a BG-11 medium containing 1 μg/mL aTc and shake-cultured for 5 days under conditions of a light quantity of 100 μmol/m2/s and 30° C. The sIr0688-suppressed strain of Example 2 and the control strain of Comparative Example 1 were also cultured under the same conditions as in Example 1.

### (3-2) Preparation of Ultrathin Section of Bacterial Strain

The culture solution obtained in the (3-1) was centrifuged at 2,500 g at room temperature for 10 minutes to retrieve the cells of the sIr1841-suppressed strain of Example 1. Subsequently, the cells were rapidly frozen with liquid propane of −175° C. and then fixed at −80° C. for 2 days using an ethanol solution containing 2% glutaraldehyde and 1% tannic acid. The cells thus fixed were dehydrated with ethanol, and the dehydrated cells were impregnated with propylene oxide and then immersed in a resin (Quetol-651) solution. Then, the resin was cured by still standing at 60° C. for 48 hours to embed the cells in the resin. The cells in the resin were sliced into a thickness of 70 nm using an ultramicrotome (Ultracut) to prepare an ultrathin section. This ultrathin section was stained using 2% uranium acetate and 1% lead citrate solutions to provide a transmission electron microscopy sample of the sIr1841-suppressed strain of Example 1. The sIr0688-suppressed strain of Example 2 and the control strain of Comparative Example 1 were also each subjected to the same operation as above to provide transmission electron microscopy samples.

### (3-3) Observation Under Electron Microscope

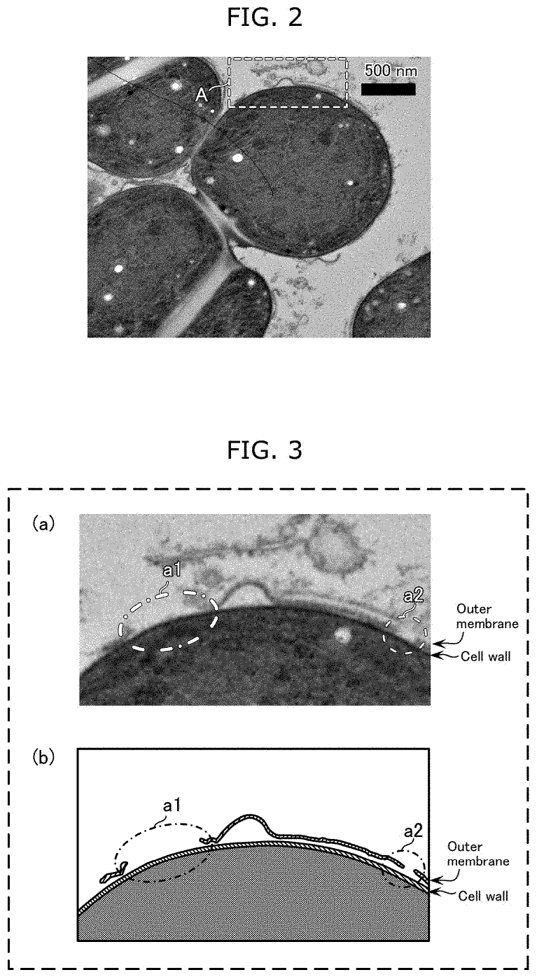

The ultrathin sections obtained in the (3-2) were observed under an accelerating voltage of 100 kV using a transmission electron microscope (JEOL JEM-1400Plus). The observation results are shown in FIGS. 2 to 7.

First, the sIr1841-suppressed strain of Example 1 will be described. FIG. 2 is a TEM (transmission electron microscope) image of the sIr1841-suppressed strain of Example 1. FIG. 3 is an enlarged image of broken line region A of FIG. 2. (a) in FIG. 3 is an enlarged TEM image of broken line region A of FIG. 2, and (b) in FIG. 3 is a diagram graphically depicting the enlarged TEM image of (a) in FIG. 3.

As illustrated in FIG. 2, in the sIr1841-suppressed strain of Example 1, the outer membrane was partially stripped (i.e., the outer membrane partially came off) from the cell wall while the outer membrane became partially loose.

In order to confirm the more detailed state of the cell surface, broken line region A was subjected to magnifying observation. As a result, as illustrated in (a) and (b) of FIG. 3, sites where the outer membrane partially came off (dot-dash line regions a1 and a2 in the figures) were able to be confirmed. Also, a site where the outer membrane became largely loose was able to be confirmed near dot-dash line region a1. This site is a site having weakened binding between the outer membrane and the cell wall. It is considered that the outer membrane swelled outward and became loose because the culture solution infiltrated into the periplasm from the outer membrane.

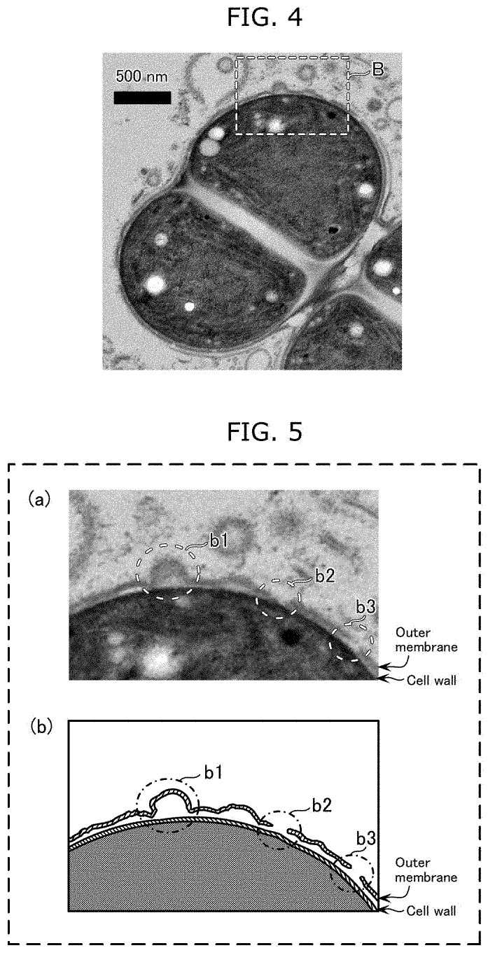

Subsequently, the sIr0688-suppressed strain of Example 2 will be described. FIG. 4 is a TEM image of the sIr0688-suppressed strain of Example 2. FIG. 5 is an enlarged image of broken line region B of FIG. 4. (a) in FIG. 5 is an enlarged TEM image of broken line region B of FIG. 4, and (b) in FIG. 5 is a diagram graphically depicting the enlarged TEM image of (a) in FIG. 5.

As illustrated in FIG. 4, in the sIr0688-suppressed strain of Example 2, the outer membrane was partially stripped from the cell wall while the outer membrane partially became loose. In the sIr0688-suppressed strain, it was able to be confirmed that the outer membrane was partially detached from the cell wall.

In order to confirm the more detailed state of the cell surface, broken line region B was subjected to magnifying observation. As a result, as illustrated in (a) and (b) in FIG. 5, a site where the outer membrane became largely loose (dot-dash line region b1 in the figures) and sites where the outer membrane partially came off (dot-dash line regions b2 and b3 in the figures) were able to be confirmed. Also, a site where the outer membrane was detached from the cell wall was able to be confirmed near each of dot-dash line regions b1, b2, and b3.

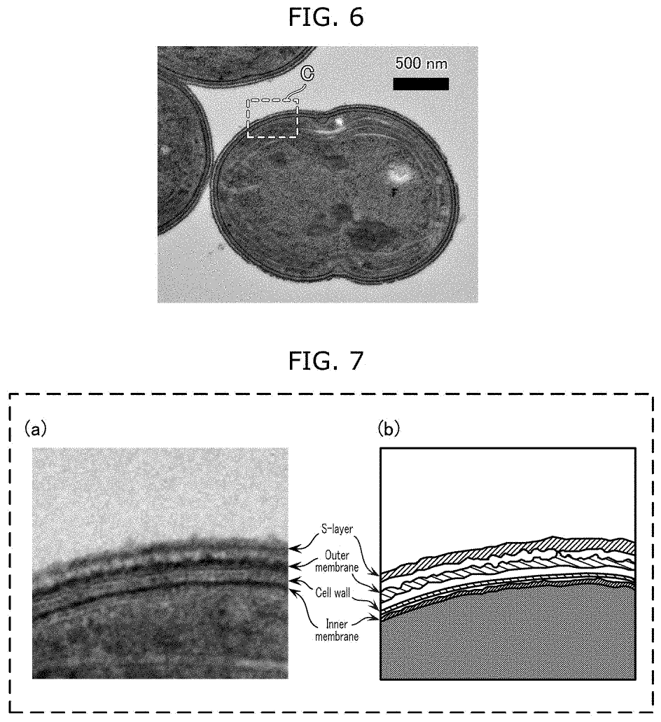

Subsequently, the control strain of Comparative Example 1 will be described. FIG. 6 is a TEM image of the control strain of Comparative Example 1. FIG. 7 is an enlarged image of broken line region C of FIG. 6. (a) in FIG. 7 is an enlarged TEM image of broken line region C of FIG. 6, and (b) in FIG. 7 is a diagram graphically depicting the enlarged TEM image of (a) in FIG. 7.

As illustrated in FIG. 6, the control strain of Comparative Example 1 had an ordered cell surface where the inner membrane, the cell wall, the outer membrane, and the S-layer were kept in a state layered in order. Specifically, the control strain exhibited none of the site where the outer membrane was detached from the cell wall, the site where the outer membrane was stripped (i.e., came off) from the cell wall, and the site where the outer membrane became loose, which were found in Examples 1 and 2.

### (4) Protein Secretory Productivity Test

The sIr1841-suppressed strain of Example 1, the sIr0688-suppressed strain of Example 2, and the control strain of Comparative Example 1 were each cultured, and the amount of protein secreted to the outside of the cells (hereinafter, also referred to as the amount of secretory protein) was measured. Each of the bacterial strains described above was evaluated for the secretory productivity of protein from the amount of protein in the culture solution. The secretory productivity of protein refers to the ability to produce protein by secreting intracellularly produced protein to the outside of the cells. Hereinafter, a specific method will be described.

### (4-1) Culture of Bacterial Strain

The sIr1841-suppressed strain of Example 1 was cultured in the same manner as in the (3-1). The culture was performed three independent times. The bacterial strains of Example 2 and Comparative Example 1 were also cultured under the same conditions as in the bacterial strain of Example 1.

### (4-2) Quantification of Protein Secreted to Outside of Cell

Each culture solution obtained in the (4-1) was centrifuged at 2,500 g at room temperature for 10 minutes to obtain a culture supernatant. The obtained culture supernatant was filtered through a membrane filter having a pore size of 0.22 μm to completely remove the cells of the sIr1841-suppressed strain of Example 1. The amount of total protein contained in the culture supernatant thus filtered was quantified by the BCA (bicinchoninic acid) method. This series of operations was performed as to each of the three culture solutions obtained by culture performed three independent times to determine a mean and standard deviation of the amounts of protein secreted to the outside of the cells of the sIr1841-suppressed strain of Example 1. The protein in the three culture solutions were also quantified under the same conditions as above as to each of the bacterial strains of Example 2 and Comparative Example 1, and a mean and standard deviation of the amounts of protein in the three culture solutions was determined.

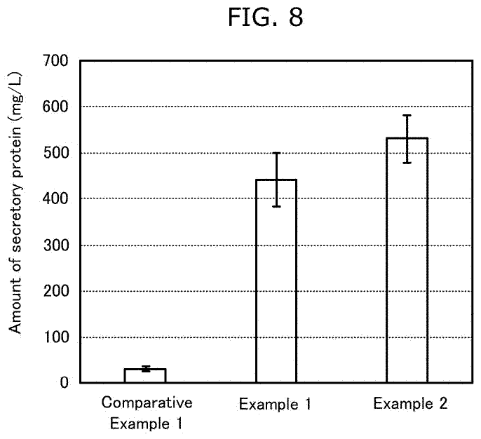

The results are shown in FIG. 8. FIG. 8 is a graph illustrating the amount of protein (n=3, error bar=SD) in the culture supernatant of a modified cyanobacterium in Example 1, Example 2, and Comparative Example 1.

As illustrated in FIG. 8, the amount (mg/L) of protein secreted into the culture supernatant was improved by approximately 25 times in all the sIr1841-suppressed strain of Example 1 and the sIr0688-suppressed strain of Example 2 compared with the control strain of Comparative Example 1.

Although the description of data is omitted, the absorbance (730 nm) of the culture solution was measured and the amount of secretory protein per g of bacterial cell dry weight (mg protein/g cell dry weight) was calculated. As a result, the amount of secretory protein per g of bacterial cell dry weight (mg protein/g cell dry weight) was improved by approximately 36 times in all the sIr1841-suppressed strain of Example 1 and the sIr0688-suppressed strain of Example 2 compared with the control strain of Comparative Example 1.

Furthermore, as illustrated in FIG. 8, the amount of protein secreted into the culture supernatant was larger for the sIr0688-suppressed strain of Example 2 in which the expression of the gene encoding the cell wall-pyruvic acid modifying enzyme (sIr0688) was suppressed than for the sIr1841-suppressed strain of Example 1 in which the expression of the gene encoding the SLH domain-containing outer membrane protein (sIr1841) was suppressed. This is probably related to a larger number of covalently linked sugar chains on cell wall surface than the number of the SLH domain-containing outer membrane protein (SIr1841) in the outer membrane. Specifically, the amount of protein secreted was increased from that for the sIr1841-suppressed strain of Example 1 probably because the sIr0688-suppressed strain of Example 2 had smaller binding level and binding force between the outer membrane and the cell wall than those of the sIr1841-suppressed strain of Example 1.

From these results, it was able to be confirmed that a function of a protein related to binding between an outer membrane and a cell wall is suppressed, whereby the binding between the outer membrane and the cell wall of cyanobacterium is partially weakened so that the outer membrane is partially detached from the cell wall. It was also able to be confirmed that the weakened binding between the outer membrane and the cell wall facilitates leaking intracellularly produced protein of cyanobacterium to the outside of the cell. Thus, the modified cyanobacterium and the production method thereof according to the present embodiment was found to largely improve the secretory productivity of protein.

### (5) Identification of Secreted Protein

Subsequently, the secreted protein contained in the culture supernatant obtained in the (4-2) was identified by LC-MS/MS. A method will be described below.

### (5-1) Sample Preparation

Cold acetone was added in an amount of 8 times the fluid volume of the culture supernatant, and the mixture was left standing at 20° C. for 2 hours and then centrifuged at 20,000 g for 15 minutes to obtain a precipitate of the protein. To this precipitate, 100 mM Tris pH 8.5 and 0.5% sodium dodecanoate (SDoD) were added, and the protein was lysed with a closed sonicator. After adjustment of the protein concentration to 1 μg/mL, dithiothreitol (DTT) (final concentration: 10 mM) was added thereto, and the mixture was left standing at 50° C. for 30 minutes. Subsequently, iodoacetamide (IAA) (final concentration: 30 mM) was added thereto, and the mixture was left standing at room temperature (in the dark) for 30 minutes. In order to terminate the reaction of IAA, cysteine (final concentration: 60 mM) was added thereto, and the mixture was left standing at room temperature for 10 minutes. 400 ng of trypsin was added thereto, and the mixture was left standing overnight at 37° C. to convert the protein to peptide fragments. After addition of 5% TFA (trifluoroacetic acid), the mixture was centrifuged at 15,000 g at room temperature for 10 minutes to obtain a supernatant. By this operation, SDoD was removed. The sample was desalted using a C18 spin column and then dried with a centrifugal evaporator. Then, 3% acetonitrile and 0.1% formic acid were added thereto, and the sample was lysed using a closed sonicator. The peptide concentration was adjusted to 200 ng/μL.

### (5-2) LC-MS/MS Analysis

The sample obtained in the (5-1) was analyzed using an LC-MS/MS apparatus (UltiMate 3000 RSLCnano LC System) under the following conditions.

Amount of sample injected: 200 ng

Column: CAPCELL CORE MP 75 μm×250 mm

Solvent: solvent A: 0.1% aqueous formic acid solution, solvent B: 0.1% formic acid+80% acetonitrile

Gradient program: 8% solvent B 4 minutes after sample injection, 44% solvent B 27 minutes later, 80% solvent B 28 minutes later, and completion of measurement 34 minutes later.

### (5-3) Data Analysis

The obtained data was analyzed under the following conditions to perform protein and peptide identification and the calculation of quantification values.

Software: Scaffold DIA

Database: UniProtKB/Swiss Prot database (Synechocystis sp. PCC 6803)

Fragmentation: HCD

Precursor Tolerance: 8 ppm

Fragment Tolerance: 10 ppm

Data Acquisition Type: Overlapping DIA

Peptide Length: 8-70

Peptide Charge: 2-8

Max Missed Cleavages: 1

Fixed Modification: Carbamidomethylation

Peptide FDR: 1% or less

Among the identified proteins, 10 types of proteins are described in Table 4 in the descending order of relative quantification values from the largest one.

TABLE 4

| | Protein name | Uniprot Accession ID | Gene name |
|---|---|---|---|
| 1 | Carboxyl-terminal protease | P73458 | prc |
| 2 | Ssr 1853 protein | P72639 | ssr1853 |
| 3 | Sl10314 protein | Q55648 | sl10314 |
| 4 | Sl10858 protein | P73742 | sl10858 |
| 5 | Sl10319 protein | P74789 | sl10319 |
| 6 | General secretion pathway protein G | P73704 | hofG |
| 7 | Slr0581 protein | Q55408 | slr0581 |
| 8 | Uncharacterized WD repeat-containing protein sl11491 | P74598 | sl11491 |
| 9 | Iron uptake protein A2 | Q55835 | futA2 |
| 10 | Plastocyanin | P21697 | petE |

All the 10 types of proteins were contained in each of the culture supernatants of the sIr1841-suppressed strain of Example 1 and the sIr0688-suppressed strain of Example 2. All of these proteins retained a periplasm (which refers to the space between the outer membrane and the inner membrane) localization signal. From these results, it was able to be confirmed that in the modified strains of Example 1 and Example 2, the outer membrane is partially detached from the cell wall, whereby protein in the periplasm easily leaks out to the outside of the outer membrane (i.e., to the outside of the bacterial cell). Thus, the modified cyanobacterium according to the present embodiment was found to have drastically improved secretory productivity of protein.

### (6) Discussion

In the present working examples, the modified cyanobacterium of the present disclosure was able to be confirmed to secrete protein present in the bacterial cell (here, in the periplasm) to the outside of the bacterial cell. The modified cyanobacterium of the present disclosure may be genetically modified, for example, so as to produce another protein instead of the proteins identified above (i.e., protein supposed to be produced within the bacterial cell), and is therefore capable of efficiently producing the desired protein. Cyanobacterium, because of its high photosynthetic ability, can be cultured by the addition of light, water, air, and trace inorganic substances to conveniently obtain necessary protein when needed, and therefore eliminates the need of using a complicated apparatus for protein production. Protein easily loses its function, for example, when processed into supplements. Hence, the modified cyanobacterium of the present disclosure can provide protein with its function maintained. Because of these advantages, the modified cyanobacterium of the present disclosure is expected to be applied to various fields.

From “(3-3) Observation under electron microscope”, it was able to be confirmed in the modified strain in which the expression of the gene encoding the protein related to the binding between the outer membrane and the cell wall was suppressed that the outer membrane partially came off by the weakened binding between the outer membrane and the cell wall; and the outer membrane partially swelled. This is probably because the culture solution easily flowed, from the outside of the cell, through osmotic pressure into a location where the outer membrane was partially detached from the cell wall by the weakened binding between the outer membrane and the cell wall; and the outer membrane partially swelled due to the culture solution that flowed therein. Since the protein concentration of the culture solution differed largely from the protein concentration in the periplasm, it is considered that the culture solution flowed into the periplasm from the outer membrane without stopping and the outer membrane that no longer withstood the internal pressure was disrupted and came off (i.e., stripped).

Among all the proteins identified by LC-MS/MS analysis, only 10 types of proteins are described in the descending order of relative quantification values from the largest one in Table 3. “(5-3) Data analysis” states that all the 10 types of proteins described in Table 3 were contained in each of the culture supernatants of the sIr1841-suppressed strain of Example 1 and the sIr0688-suppressed strain of Example 2. It was also confirmed that all the other proteins identified by LC-MS/MS analysis were contained in each of the culture supernatants of Example 1 and Example 2. Further, the secreted proteins were confirmed to include protein supposed to be present in the periplasm, and protein that is transported from the cytoplasm to the periplasm and functions in the periplasm.

Although a modified cyanobacterium, a modified cyanobacterium production method, and a protein production method according to the present disclosure are described above based on the foregoing embodiments, the present discloser is not limited by the foregoing embodiments. Forms obtained by making various modifications to the foregoing embodiments that can be conceived by those skilled in the art as well as other forms realized by combining some of the components in the foregoing embodiments, for as long as they do not depart from the essence of the present disclosure, are included in the scope of the present disclosure.

In the embodiments described above, examples are described in which protein produced within the bacterial cell leaks out to the outside of the bacterial cell by suppressing or losing a function of a protein involved in binding between an outer membrane and a cell wall of cyanobacterium. However, the present disclosure is not limited thereby. For example, the binding between the outer membrane and the cell wall may be weakened or the outer membrane may be rendered fragile by applying external force to cyanobacterium. Alternatively, the outer membrane may be rendered fragile by adding an enzyme or a drug to the culture solution of cyanobacterium.

### INDUSTRIAL APPLICABILITY

The modified cyanobacterium, the modified cyanobacterium production method, and the protein production method using the modified cyanobacterium according to the present disclosure can efficiently produce protein by culturing the modified cyanobacterium by the addition of water, light, air, and trace inorganic substances. For example, an enzyme for food ingredient starting material or compound production, a diagnostic or therapeutic enzyme in the medical field, or an enzyme for feed in the agricultural, fishery, and livestock fields can be obtained.

1. A modified cyanobacterium in which a function of a protein involved in binding between an outer membrane and a cell wall of cyanobacterium is suppressed or lost.

2. The modified cyanobacterium according to claim 1, wherein
the protein involved in the binding between the outer membrane and the cell wall is at least one of a surface layer homology (SLH) domain-containing outer membrane protein or a cell wall-pyruvic acid modifying enzyme.

3. The modified cyanobacterium according to claim 2, wherein
the SLH domain-containing outer membrane protein is:
SIr1841 having an amino acid sequence represented by SEQ ID NO: 1;
NIES970_09470 having an amino acid sequence represented by SEQ ID NO: 2;
Anacy_3458 having an amino acid sequence represented by SEQ ID NO: 3; or
a protein having an amino acid sequence that is at least 50 percent identical to the amino acid sequence of any one of the SIr1841, the NIES970_09470, and the Anacy_3458.

4. The modified cyanobacterium according to claim 2, wherein
the cell wall-pyruvic acid modifying enzyme is:
SIr0688 having an amino acid sequence represented by SEQ ID NO: 4;
Synpcc7942_1529 having an amino acid sequence represented by SEQ ID NO: 5;
Anacy_1623 having an amino acid sequence represented by SEQ ID NO: 6; or
a protein having an amino acid sequence that is at least 50 percent identical to the amino acid sequence of any one of the SIr0688, the Synpcc7942_1529, and the Anacy_1623.

5. The modified cyanobacterium according to claim 1, wherein
a gene which causes expression of the protein involved in the binding between the outer membrane and the cell wall is deleted or inactivated.

6. The modified cyanobacterium according to claim 5, wherein
the gene which causes expression of the protein involved in the binding between the outer membrane and the cell wall is at least one of a gene encoding an SLH domain-containing outer membrane protein or a gene encoding a cell wall-pyruvic acid modifying enzyme.

7. The modified cyanobacterium according to claim 6, wherein
the gene encoding the SLH domain-containing outer membrane protein is:
sIr1841 having a nucleotide sequence represented by SEQ ID NO: 7;
nies970_09470 having a nucleotide sequence represented by SEQ ID NO: 8;
anacy_3458 having a nucleotide sequence represented by SEQ ID NO: 9; or
a gene having a nucleotide sequence that is at least 50 percent identical to the nucleotide sequence of any one of the sIr1841, the nies970_09470, and the anacy_3458.

8. The modified cyanobacterium according to claim 6, wherein
the gene encoding the cell wall-pyruvic acid modifying enzyme is:
sIr0688 having a nucleotide sequence represented by SEQ ID NO: 10;
synpcc7942_1529 having a nucleotide sequence represented by SEQ ID NO: 11;
anacy_1623 having a nucleotide sequence represented by SEQ ID NO: 12; or
a gene having a nucleotide sequence that is at least 50 percent identical to the nucleotide sequence of any one of the sIr0688, the synpcc7942_1529, and the anacy_1623.

9. A modified cyanobacterium production method comprising:
causing a function of a protein involved in binding between an outer membrane and a cell wall of cyanobacterium to be suppressed or lost.

10. A protein production method comprising:
culturing the modified cyanobacterium according to claim 1.
